(19) United States
(12) Patent Application Publication
(10) Pub. No.: US 2026/0076666 A1
(43) Pub. Date: Mar. 19, 2026

(54) SUTURE ANCHOR SYSTEM AND METHOD OF USE

(71) Applicant: Skeletal Dynamics, Inc., Miami, FL (US)

(72) Inventors: Jorge L. Orbay, Miami, FL (US); Gabriel Mesa, Miami, FL (US); Daniel Sixto, Miami, FL (US); Adelbert Mencias, South bend, IN (US)

(21) Appl. No.: 19/324,572

(22) Filed: Sep. 10, 2025

Related U.S. Application Data
(60) Provisional application No. 63/696,702, filed on Sep. 19, 2024.

(51) Int. Cl. A61B 17/04

(52) U.S. Cl. CPC A61B 17/0401; A61B 2017/0409; A61B 2017/0414; A61B 2017/0424; A61B 2017/044; A61B 2017/0445; A61B 2017/0453

(57) ABSTRACT

A suture anchor system for securing a suture to a bone, the system comprising an anchor liner and an inner locking component; the anchor liner comprising a proximal end, a distal end, and a substantially cylindrical elongated body with a longitudinal axis; the body having an external thread, a cavity along the longitudinal axis with an inner wall and an opening at the proximal end, a suture hole transverse to the longitudinal axis near the distal end, and one or more expansion areas through a portion of the body at the proximal end; the inner locking component comprising a leading end, a trailing end, and an elongated body with an outer surface adapted to engage the cavity inner wall.

### CLAIM OF PRIORITY

This application is being filed as a non-provisional patent application under 35 U.S.C. § 111(a) and 37 CFR § 1.53(b). This application claims priority under 35 U.S.C. § 119(e) to U.S. provisional patent application Ser. No. 63/696,702 filed on Sep. 19, 2024, the contents of which are incorporated herein by reference.

### TECHNICAL FIELD

The technical field relates generally to surgical devices and systems and, in particular, to anchors for fixation of sutures to bones, soft tissue, and other structures.

### BACKGROUND

The detachment of soft tissue, such as ligaments or tendons, from the bone is a common injury. In some cases, surgery is required to reattach the soft tissue to the bone. One method of reattaching the soft tissue involves the use of a suture anchor or bone anchor. Many procedures can benefit from the use of suture anchors, such as in attaching soft tissue to a bone, securing a suture for bone repair including in repairing fractures, stabilizing joints and bone union, securing an orthopedic plate, screw or other structure to a bone, etc. The suture anchor may secure various types of sutures, such as absorbable (biodegradable) and non-absorbable sutures, monofilament and multifilament sutures, and natural and synthetic sutures, can benefit from anchors.

Suture or bone anchors are generally composed of an anchor body that is inserted into the bone, a suture that connects the bone and soft tissue (or other bones or structures), and an eyelet that interfaces between the two. The suture or bone anchor acts to anchor the suture to the bone in order to rebuild the connection between the bone and tissue (or other bones or structures.).

Maintaining a stable and strong connection between the tissue (or other structures, including bones) and bone through the connecting suture is important in restoring functionality to the injured site. However, poor stability in a suture or suture anchor may cause it to loosen over time and, in some cases, the bone may be too weak to hold the suture anchor in place causing it to fail. If the suture anchor loosens or fails, then the connecting suture may also loosen or even become detached.

Therefore, there exists a need for a suture anchor that can maintain tension and stability in securing a suture to a bone.

### SUMMARY

It is among the objects of the present system and method to overcome the limitations of the heretofore-known devices and methods by providing inventive features to achieve: a.) a suture anchor with increased stability and strength; b.) adjustable tensioning of a suture pre-insertion and post-insertion into the bone; c.) maintained tension in the suture over an extended period; and d.) lower risk of suture anchor failure and suture detachment.

It is also an object of the present system to provide a suture anchor adapted for securing a suture to a bone. The suture anchor system may comprise an anchor liner and an inner locking component. In at least one embodiment, the anchor liner may comprise a substantially cylindrical elongated body having a proximal end, a distal end, and a longitudinal axis therebetween.

The body of the anchor liner may comprise an external thread on an exterior surface of the body. The body may comprise a cavity bored through the body extending from the proximal end along the longitudinal axis. The cavity may have an inner wall and an opening at the proximal end of the anchor liner. In some embodiments, the inner wall of the cavity may optionally comprise an internal thread. In other embodiments, the inner wall of the cavity may optionally be smooth or textured. In at least one embodiment, the cavity of the anchor liner may be further adapted to receive a driving tip of an insertion tool.

The body of the anchor liner may also comprise a suture hole through the body transverse to the longitudinal axis and near the distal end of the anchor liner. In some embodiments, the body may optionally further comprise first and second suture channels parallel to the longitudinal axis along opposite sides of the body. In such embodiments, each suture channel may be recessed in the external thread of the body and may extend from the proximal end of the anchor liner to a respective opening of the suture hole. In at least one embodiment, the suture hole, the first suture channel, and the second suture channel may be adapted to receive the suture.

In some embodiments, the exterior surface of the body of the anchor liner may optionally comprise a threaded section and a smooth section. In such embodiments, the threaded section may extend from the proximal end of the anchor liner partially along the body and may comprise the external thread of the body. In further such embodiments, the smooth section may comprise the suture hole and extend from the distal end of the anchor liner to the threaded section.

In further embodiments, the body of the anchor liner may comprise one or more expansion areas through a portion of the body at the proximal end. In some embodiments, the one or more expansion areas may optionally comprise slits, expansion channels, slices, weakened or thinned areas, elastic areas, gaps, or perforated areas. In embodiments having first and second suture channels on the body of the anchor liner, each of the one or more expansion areas may be located radially between the first and second suture channels.

The inner locking component of the suture anchor system may comprise a leading end, a trailing end and an elongated body therebetween. The trailing end may be adapted to receive a driving tool. The body of the inner locking component may have an outer surface adapted to engage with the inner wall of the cavity. In at least some embodiments, the outer surface of the inner locking component may optionally further comprise an external thread. In other embodiments, the outer surface of the inner locking component may optionally be smooth or textured.

In some embodiments, the outer surface of the inner locking component further comprises an external thread and the inner wall of the cavity of the anchor liner comprises an internal thread. In such embodiments, the external thread of the outer surface may be adapted to engage the internal thread of the inner wall.

In further embodiments, the outer surface of the inner locking component comprises an external thread and the inner wall of the cavity is smooth or textured. In such embodiments, the external thread of the outer surface may be adapted to engage the inner wall.

In other embodiments, the outer surface of the inner locking component is smooth or textured and the inner wall of the cavity of the anchor liner comprises an internal thread. In such embodiments, the internal thread may be adapted to engage the outer surface.

In further embodiments, the outer surface of the inner locking component is textured and the inner wall of the cavity is textured. In such embodiments, the textured outer surface may be adapted to engage the textured inner wall.

In at least one embodiment, the inner locking component may optionally further comprise a head at the trailing end. In other embodiments, the inner locking component may optionally be headless at the trailing end. In some embodiments, the body of the inner locking component may optionally be substantially cylindrical. In further embodiments, the body of the inner locking component may optionally be tapered or frustoconical in shape with a diameter narrowing gradually from the trailing end to the leading end.

In at least one embodiment, the proximal end of the anchor liner may be adapted to progressively expand radially as the inner locking component is inserted into the cavity. In further embodiments, the anchor liner may be adapted to lock the suture between the bone and the anchor liner as the proximal end of the anchor liner expands.

In at least one further embodiment, the external thread of the anchor liner may optionally be adapted to engage the bone through rotation. In other embodiments, the external thread of the anchor liner may optionally be adapted to engage the bone through longitudinal force. In at least one embodiment, the external thread of the anchor liner may optionally be adapted to engage the bone through either rotation or longitudinal force.

A further object is to provide an insertion tool adapted for inserting an anchor liner of a suture anchor system into a bone and tensioning a suture therein. In at least one embodiment, the insertion tool may comprise a proximal end, a distal end and a body with a longitudinal axis therebetween. The body may optionally further comprise a handle and an elongated shaft extending from the handle to the distal end. The handle may have a suture retention mechanism. The distal end of the insertion tool may comprise a driving tip extending distally outward. The driving tip may be adapted to be received by the anchor liner of the suture anchor system.

In at least some embodiments, the suture retention mechanism may optionally comprise two arms each with a slit extending transverse to the longitudinal axis. In such embodiments, the two arms may optionally be rotatably engaged with the body. In some embodiments having two arms, each slit on each of the two arms may be adapted to receive a respective end of the suture for tensioning. In further embodiments having two arms, each of the two arms may be adapted to receive an instrument for tensioning the suture.

In at least one embodiment, the driving tip may optionally be further adapted to drive the anchor liner into the bone. In such embodiments, the driving tip may be adapted to drive the anchor liner into the bone through rotation and/or longitudinal force.

In at least some embodiments, the shaft may optionally further comprise first and second channels through the shaft. In such embodiments, each channel may extend from an opposite side of the shaft to a respective first suture hole and second suture hole. In some embodiments, the first suture hole and second suture hole may be located at the distal end of the insertion tool. In embodiments having a first suture hole and second suture hole, the driving tip may optionally be located between the first and second suture holes at the distal end of the insertion tool.

In embodiments having first and second suture holes and first and second channels, the first suture hole and the second suture hole may be adapted to align with respective first and second suture channels of the anchor liner. In further embodiments, the first suture hole and the first channel of the insertion tool may be adapted to receive an end of the suture. The second suture hole and the second channel may also be adapted to receive an end of the suture. In other embodiments, the first and second channels and the respective first and second suture holes may be adapted to maintain alignment of the suture when inserting the anchor liner into the bone.

In other embodiments, the shaft of the body may optionally comprise first and second channels along an exterior of the shaft. In such embodiments, each channel may be on opposite sides of the shaft extending from the distal end and be adapted to align with respective first and second suture channels of the anchor liner.

In further embodiments, the insertion tool may optionally further comprise a suture alignment clip. The suture alignment clip may comprise a substantially cylindrical hollow body having a proximal end, a distal end and a length therebetween, an opening slit extending from the proximal end to the distal end for the length of the body and having a width, first and second side slits extending from the proximal end partially along the length of the body and located on opposite sides of the body, and first and second protrusions on an interior of the body located on opposite sides of the body and extending from the distal end partially along the length of the body to the respective first and second side slits. The first protrusion may align with the first side slit and the second protrusion may align with the second side slit. The suture alignment clip may attach to the shaft at the distal end of the insertion tool. In such embodiments, the first and second protrusions may be adapted to align with and slide into respective first and second channels on the shaft of the insertion tool.

In at least some embodiments, the first and second protrusions are each adapted to compress a suture received by each first and second suture channels of the insertion tool, and the first and second side slits are each adapted to allow an end of the suture to exit the suture alignment clip when the suture alignment clip is attached to the shaft at the distal end of the insertion tool.

In at least one embodiment, the insertion tool may optionally further comprise a gripping member at the proximal end. In at least one further embodiment, the insertion tool may be adapted to be used in conjunction with a hammer or a mallet to drive the anchor liner into the bone without screwing it. In further embodiments, the proximal end of the insertion tool may further comprise a surface adapted to receive a longitudinal force from the hammer or the mallet.

Although the system, tool and method are illustrated and described herein as embodied for use in securing a suture to a bone, it is nevertheless not intended to be limited to only the details shown, since various modifications and structural changes may be made therein without departing from the spirit of the system and method and within the scope and range of equivalents of the claims.

The construction of the system, tool and method, however, together with additional objects and advantages thereof will be best understood from the following description of the specific disclosed embodiments when read in connection with the accompanying drawings.

### BRIEF DESCRIPTION OF THE DRAWINGS

FIG. 1 illustrates a perspective view of a suture anchor system comprising an anchor liner and an inner locking component, the suture anchor system having received a suture in accordance with one embodiment.

FIG. 2 illustrates an exploded view of a suture anchor system and a suture before an anchor liner of the suture anchor system has received an inner locking component in accordance with one embodiment.

FIG. 3 illustrates an exploded view of the suture anchor system and suture of FIG. 1 after a proximal end of the anchor liner has expanded from the anchor liner having received the inner locking component in accordance with one embodiment.

FIGS. 4 and 5 illustrate side and cross-sectional side views, respectively, of the suture anchor system of FIG. 1 having received a suture in accordance with one embodiment.

FIG. 6 illustrates an enlarged view of the suture anchor system of FIGS. 1, 4 and 5 showing additional details in accordance with one embodiment.

FIG. 7 illustrates a perspective view of an anchor liner in accordance with one embodiment.

FIG. 8 illustrates a cross-sectional view of the anchor liner of FIG. 7 showing additional details in accordance with one embodiment.

FIGS. 9 and 10 illustrate perspective views of the anchor liner of FIGS. 7 and 8 showing additional details in accordance with one embodiment.

FIGS. 11 and 12 illustrate side views of the anchor liner of FIGS. 7 through 10 showing additional details in accordance with one embodiment.

FIGS. 13 and 14 illustrate top and bottom views, respectively, of the anchor liner of FIGS. 7 through 12 showing additional details in accordance with one embodiment.

FIG. 15 illustrates a top perspective view of the anchor liner of FIGS. 7 through 14 showing additional details in accordance with one embodiment.

FIGS. 16 and 17 illustrate perspective views of an anchor liner after the proximal end of the anchor liner has expanded in accordance with one embodiment.

FIGS. 18 and 19 illustrate side and top views, respectively, of the anchor liner with the expanded proximal end of FIGS. 16 and 17 showing additional details in accordance with one embodiment.

FIGS. 20 and 21 illustrate perspective and side views, respectively, of an inner locking component having a head in accordance with one embodiment.

FIGS. 22 and 23 illustrate side views of an anchor liner having received an inner locking component having a head in accordance with one embodiment.

FIGS. 24 and 25 illustrate cross-sectional and partial cross-sectional views, respectively, of the anchor liner having received the inner locking component of FIGS. 22 and 23 showing additional details in accordance with one embodiment.

FIG. 26 illustrates a side view of a suture anchor system comprising an anchor liner and an inner locking component, the suture anchor system having received a suture and suture tape in accordance with one embodiment.

FIGS. 27, 28 and 29 illustrate perspective, side and top perspective views, respectively, of a headless inner locking component in accordance with one embodiment.

FIG. 30 illustrates a perspective view of a suture anchor system comprising an anchor liner and a headless inner locking component, the suture anchor system having received a suture in accordance with one embodiment.

FIG. 31 illustrates an exploded view of a suture and a suture anchor system having a headless inner locking component before an anchor liner of the suture anchor system has received the headless inner locking component in accordance with one embodiment.

FIGS. 32 and 33 illustrate side and cross-sectional side views, respectively, of the suture anchor system of FIG. 30 having received a suture in accordance with one embodiment.

FIG. 34 illustrates a perspective view of a suture anchor system comprising an anchor liner and a headless inner locking component, the anchor liner having received the headless inner locking component in accordance with one embodiment.

FIGS. 35 and 36 illustrate side views of the suture anchor system of FIG. 34, the anchor liner having received the headless inner locking component, showing additional details in accordance with one embodiment.

FIGS. 37 and 38 illustrate perspective and side views, respectively, of an insertion tool adapted for inserting an anchor liner of a suture anchor system into a bone and tensioning a suture therein in accordance with one embodiment.

FIGS. 39 and 40 illustrate enlarged perspective and enlarged cross-sectional views, respectively, of the insertion tool of FIGS. 37 and 38 showing additional details in accordance with one embodiment.

FIG. 41 illustrates a bottom perspective view of the insertion tool of FIGS. 37 and 38 showing additional details in accordance with one embodiment.

FIG. 42 illustrates an enlarged bottom view of the insertion tool of FIG. 41 showing additional details in accordance with one embodiment.

FIG. 43 illustrates an anchor liner having received a driving tip of the insertion tool, the anchor liner and the insertion tool having received a suture in accordance with one embodiment.

FIGS. 44 and 45 illustrate enlarged views of the anchor liner having received the driving tip of the insertion tool of FIG. 43, showing additional details in accordance with one embodiment. The anchor liner of FIG. 45 is illustrated as transparent for illustrative purposes only.

FIG. 46 illustrates a side view of the anchor liner inserted into a bone using the insertion tool, the anchor liner having received the driving tip of the insertion tool, the anchor liner and the insertion tool having received the suture in accordance with one embodiment. The bone, insertion tool and anchor liner are illustrated as transparent for illustrative purposes only.

FIG. 47 illustrates a perspective view the anchor liner inserted in a bone using the insertion tool, and an instrument for tensioning the suture received by an arm of the insertion tool in accordance with one embodiment.

FIG. 48 illustrates an enlarged view of the anchor liner inserted in a bone using the insertion tool of FIG. 47, showing additional details in accordance with one embodiment. The bone, insertion tool and anchor liner are illustrated as transparent for illustrative purposes only.

FIG. 49 illustrates a perspective view of a bone having received a suture anchor system and suture in accordance with one embodiment.

FIGS. 50 and 51 illustrate perspective views of an anchor liner in accordance with another embodiment.

FIGS. 52 and 53 illustrate top and bottom views, respectively, of the anchor liner of FIGS. 50 and 51 showing additional details in accordance with one embodiment.

FIG. 54 illustrates a top perspective view of the anchor liner of FIGS. 50 through 53 showing additional details in accordance with one embodiment.

FIG. 55 illustrates a cross-sectional view of the anchor liner of FIGS. 50 through 54 showing additional details in accordance with one embodiment.

FIGS. 56 through 58 illustrate perspective views of an insertion tool in accordance with another embodiment, FIG. 58 is shown as transparent for illustrative purposes only.

FIG. 59 illustrates a perspective view of a shaft of the insertion tool of FIGS. 56 through 58 showing additional details in accordance with one embodiment.

FIG. 60 illustrates an enlarged bottom view of the shaft of the insertion tool of FIGS. 56 through 59 showing additional details in accordance with one embodiment.

FIGS. 61 and 62 illustrate front and back perspective views, respectively, of the anchor liner of FIGS. 50 through 55 having received a driving tip of the insertion tool of FIGS. 56 through 60, and the insertion tool having a suture alignment clip in accordance with one embodiment.

FIGS. 63 and 64 illustrate enlarged perspective views of the anchor liner and insertion tool with suture alignment clip of FIG. 61 in accordance with one embodiment, the anchor liner and insertion tool with suture alignment clip of FIG. 63 are all shown as transparent for illustrative purposes only.

FIG. 65 illustrates a perspective view of a suture alignment clip in accordance with one embodiment.

FIG. 66 illustrates a perspective view of the suture alignment clip of FIG. 65 showing additional in accordance with one embodiment, the suture alignment clip shown as transparent for illustrative purposes only.

FIGS. 67 and 68 illustrate top and bottom views, respectively, of the suture alignment clip of FIGS. 65 and 66 in accordance with one embodiment.

FIG. 69 illustrates a perspective view of the anchor liner and insertion tool with suture alignment clip having received a suture in accordance with one embodiment.

FIG. 70 illustrates an enlarged perspective view of the anchor liner and insertion tool with suture alignment clip having received a suture of FIG. 69 showing additional details in accordance with one embodiment.

FIG. 71 illustrates an enlarged front view of the anchor liner and insertion tool with suture alignment clip having received a suture of FIG. 69 showing additional details in accordance with one embodiment, the anchor liner, insertion tool with suture alignment clip and suture all shown as transparent for illustrative purposes only.

### DETAILED DESCRIPTION

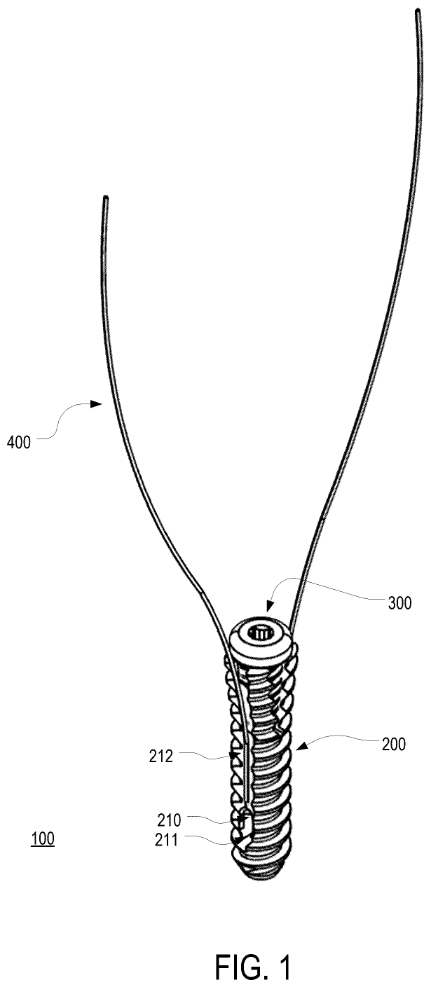

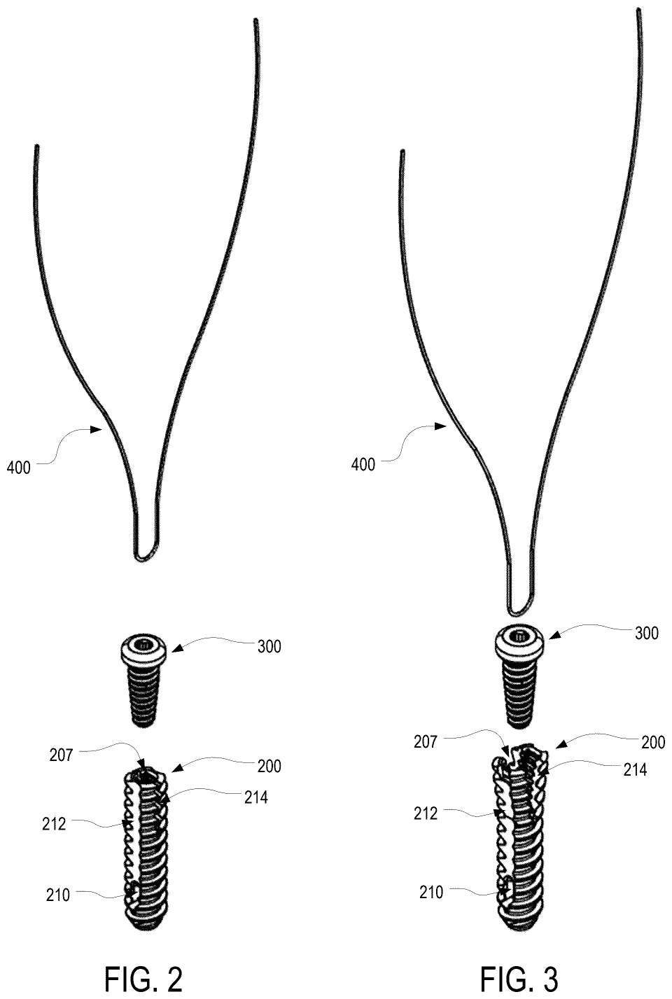

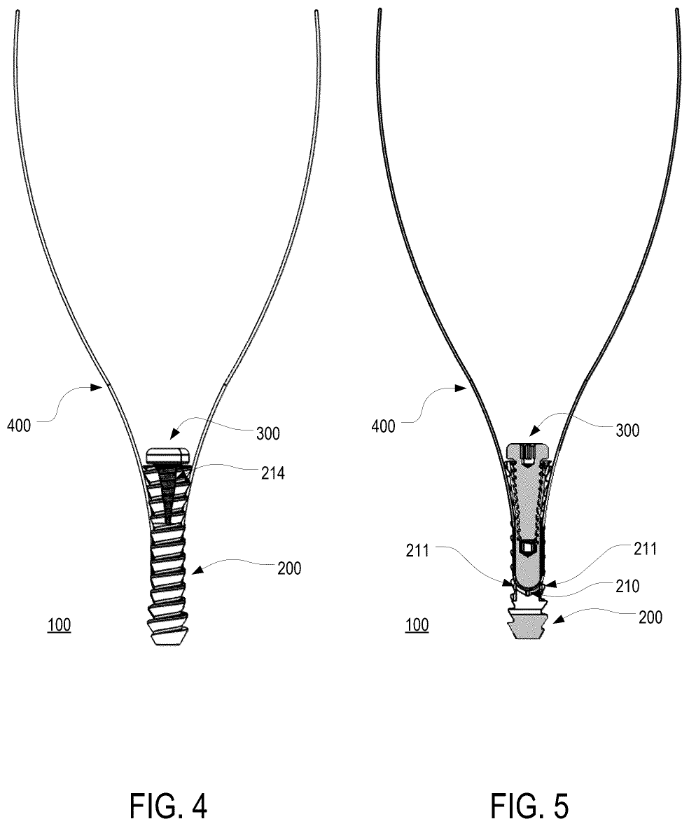

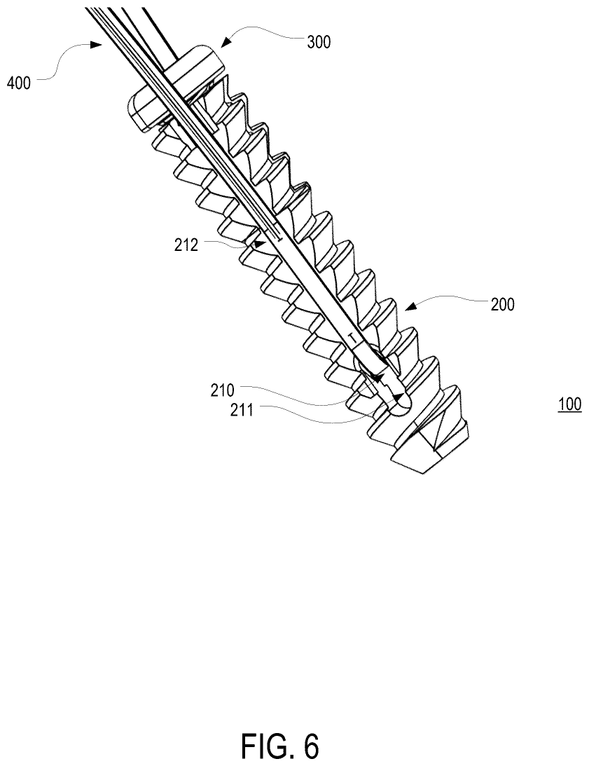

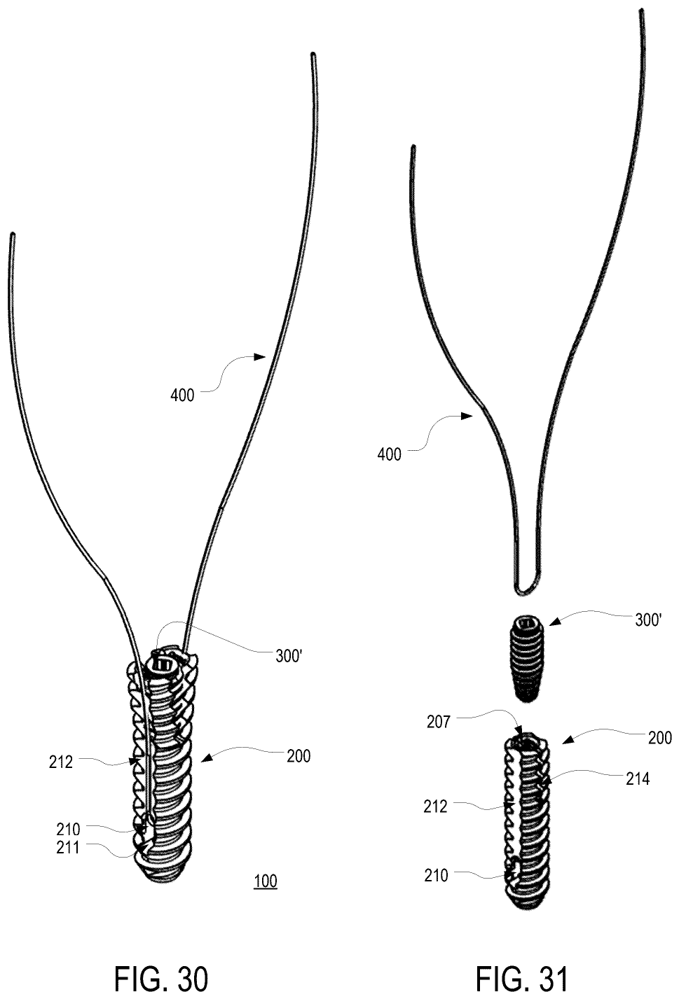

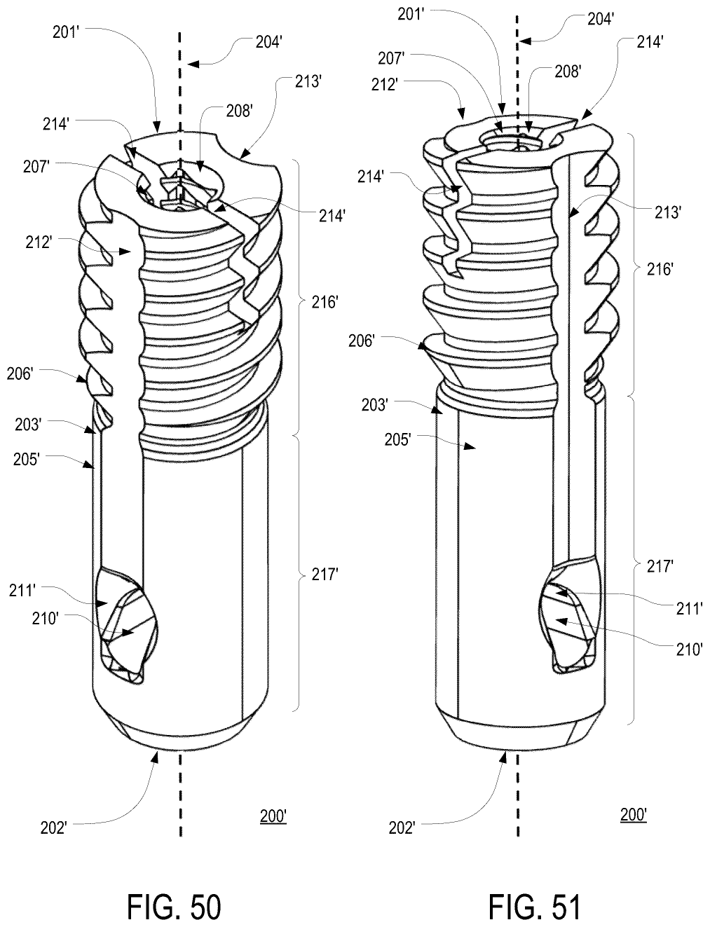

Referring first to FIGS. 1, 4 and 5, shown are perspective, side and cross-sectional views, respectively, of a suture anchor system (100) having received a suture (400) in accordance with one embodiment. FIG. 6 shows an enlarged view of the suture anchor system (100) having received the suture (400). FIG. 30 illustrates a perspective view of another embodiment of a suture anchor system (100) having received a suture (400). The suture anchor system (100) may be adapted for securing a suture (400) to a bone (800) (see FIG. 49). In at least one embodiment, the suture anchor system (100) comprises an anchor liner (200; 200′ in FIG. 50) and an inner locking component (300; 300′ in FIG. 30). Referring now to FIGS. 2 and 3, shown are exploded views of the suture anchor system (100), comprising the anchor liner (200) and one embodiment of the inner locking component (300), and suture (400). FIG. 2 illustrates an exploded view of the anchor liner (200), inner locking component (300) and suture (400) before the anchor liner (200) has received the inner locking component (300) and undergone any expansion. FIG. 3 illustrates an exploded view of the anchor liner (200), inner locking component (300) and suture (400) after the anchor liner (200) has received the inner locking component (300) and expanded at a proximal end (201) of the anchor liner (200). FIG. 31 illustrates an exploded view of the suture (400), anchor liner (200) and another embodiment of the inner locking component (300′) before the anchor liner (200) has received the inner locking component (300′) and undergone any expansion.

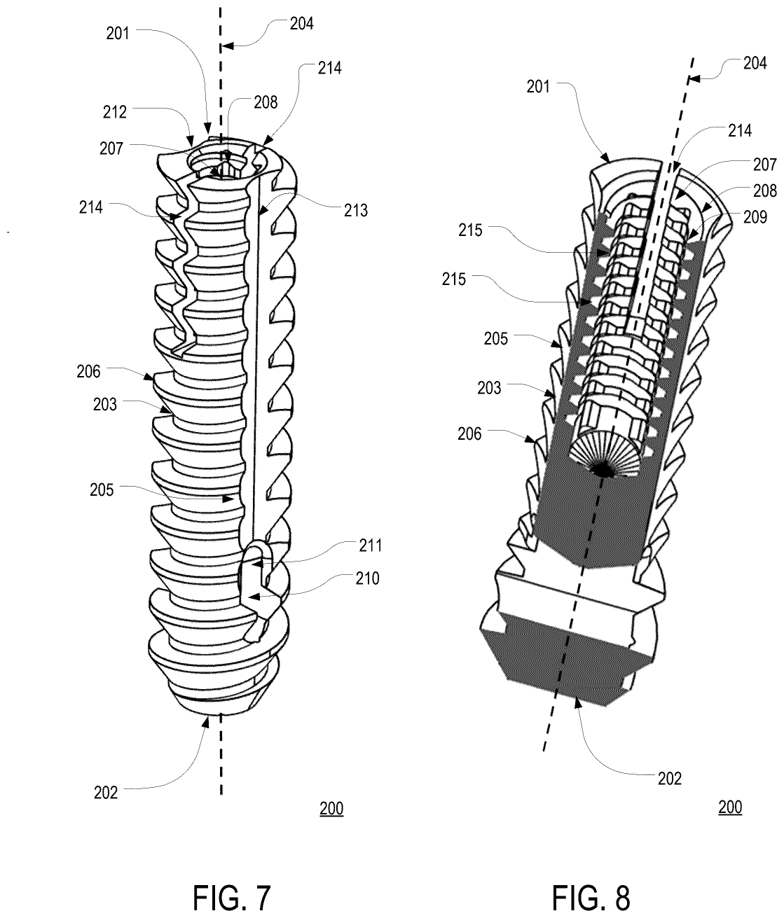

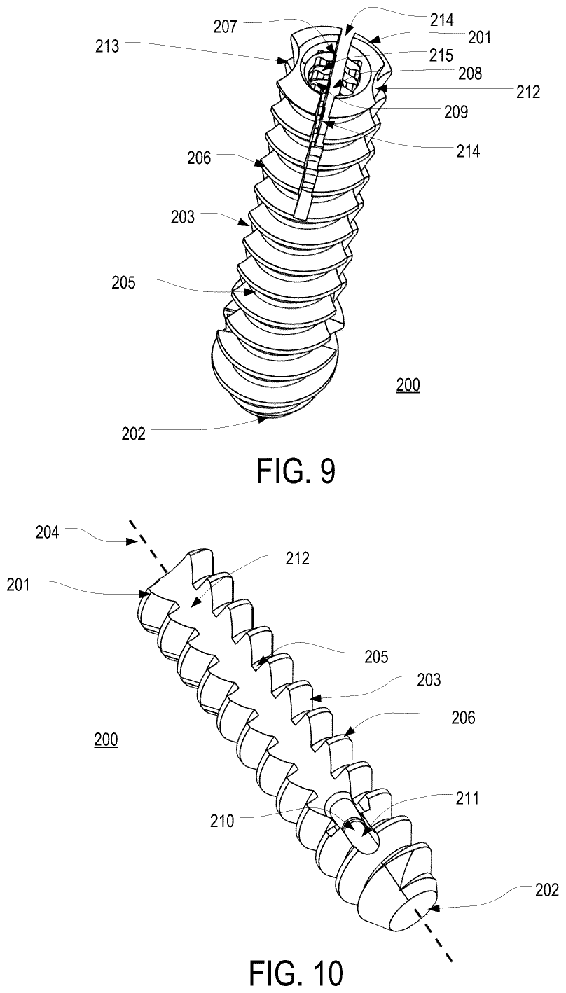

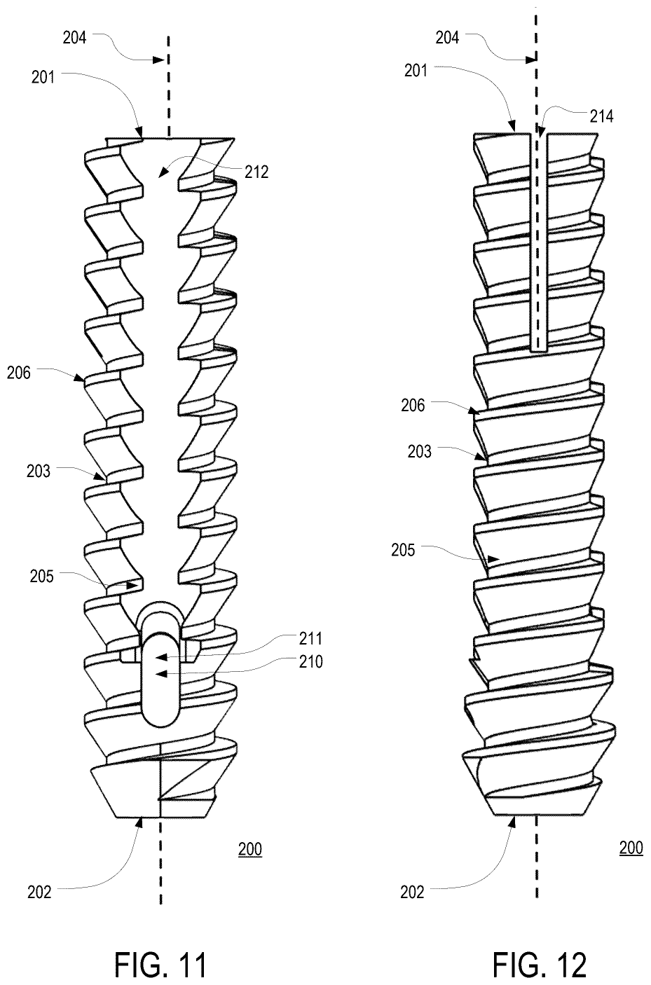

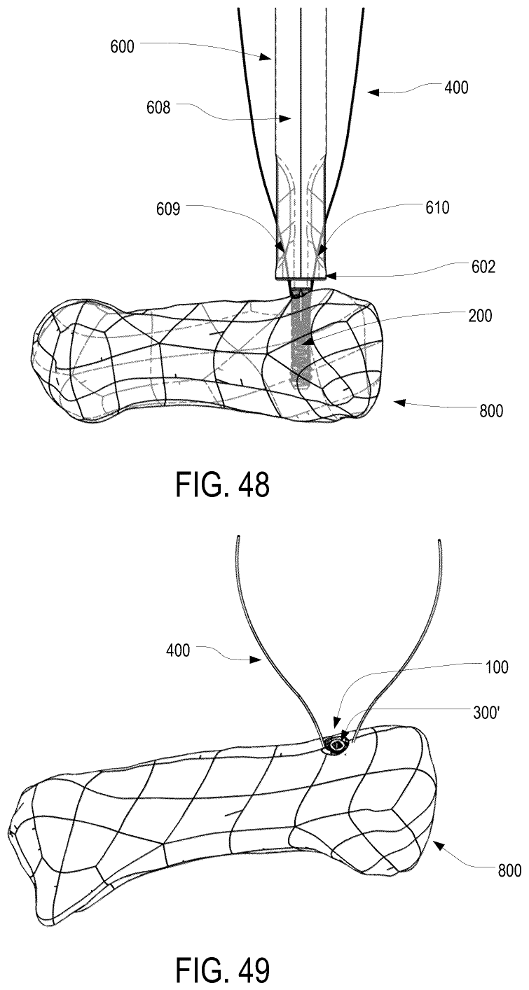

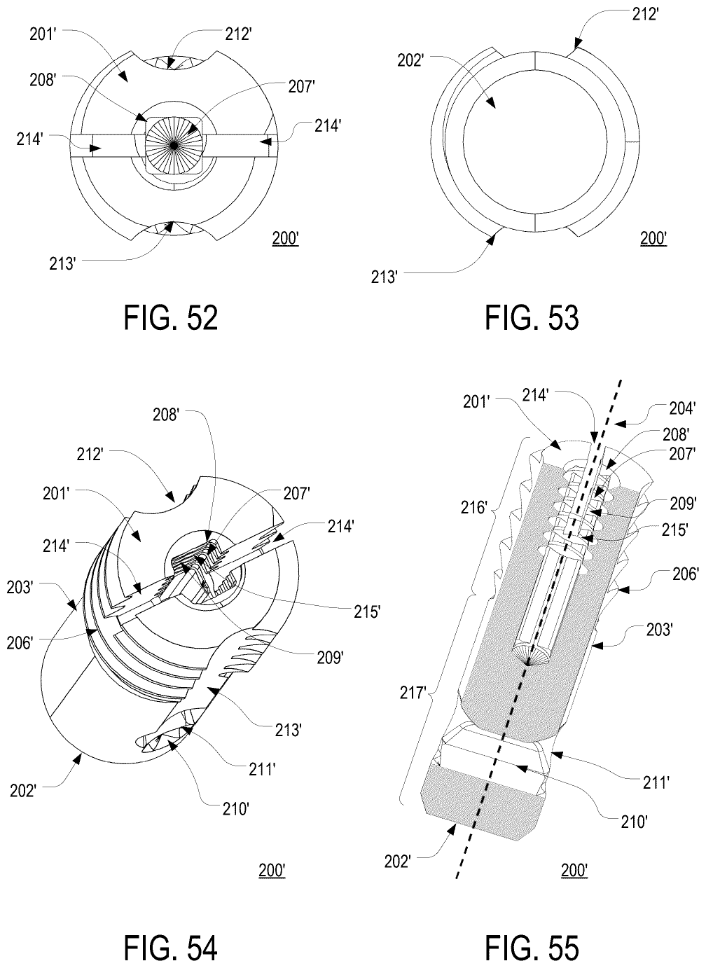

Next, referring to FIGS. 7 through 15, shown are various views of an anchor liner (200) before any expansion in accordance with at least one embodiment. FIGS. 50 through 55 show various views of an anchor liner (200′) in accordance with another embodiment. The anchor liner (200, 200′) of the system (100) may comprise a substantially cylindrical elongated body (203, 203′) having a proximal end (201, 201′), a distal end (202, 202′) and a longitudinal axis (204, 204′) therebetween. The body (203, 203′) may optionally further comprise an external thread (206, 206′) on an exterior surface (205, 205′) of the body (203, 203′). In some embodiments, such as the anchor liner (200′) depicted in FIGS. 50 through 55, the exterior surface (205′) of the body (203′) may optionally comprise a threaded section (216′) and a smooth section (217′). In such embodiments, the threaded section (216′) may comprise the external thread (206′) of the body (203′) and extends from proximal end (201′) partially along the body (203′) of the anchor liner (200′). In other embodiments, such as the anchor liner (200) depicted in FIGS. 7 through 15, the external thread (206) of the exterior surface (205) of the body (203) may extend along the entire body (203) length of the anchor liner (200). In at least one embodiment, the external thread (206, 206′) on the body (203, 203′) of the anchor liner (200, 200′) may be adapted to engage a bone (800) (see FIGS. 48 and 49). The shape of the external thread (206, 206′) is not limited to a traditional helical thread style. The shape of the external thread (206, 206′) can be adapted depending on the application for which the suture anchor system (100) is being used. For example, in some embodiments, the external thread (206, 206′) of the body (203, 203′) may optionally be a non-helical buttress thread adapted for push in and screw in insertion into the bone (800). In other embodiments, the body (203, 203′) may optionally comprise a strut, a stanchion, or an abutment that provides rigid fixation against a variety of bones.

In at least one embodiment, the external thread (206, 206′) of the anchor liner (200, 200′) may be adapted to engage the bone through rotation (engagement not shown). In further embodiments, the external thread (206, 206′) of the anchor liner (200, 200′) may be adapted to engage the bone through longitudinal force (engagement not shown). In at least some embodiments, the external thread (206, 206′) may be adapted to engage the bone through either rotation or longitudinal force (engagement not shown). FIG. 48 illustrates a bone (800) after having received the anchor liner (200) using an insertion tool (600). The bone (800), suture anchor liner (200) and insertion tool (600) in FIG. 48 are all shown as transparent for illustrative purposes only. Referring to FIG. 49, shown is the bone (800) after having received the suture anchor system (100) and the suture (400) secured therein.

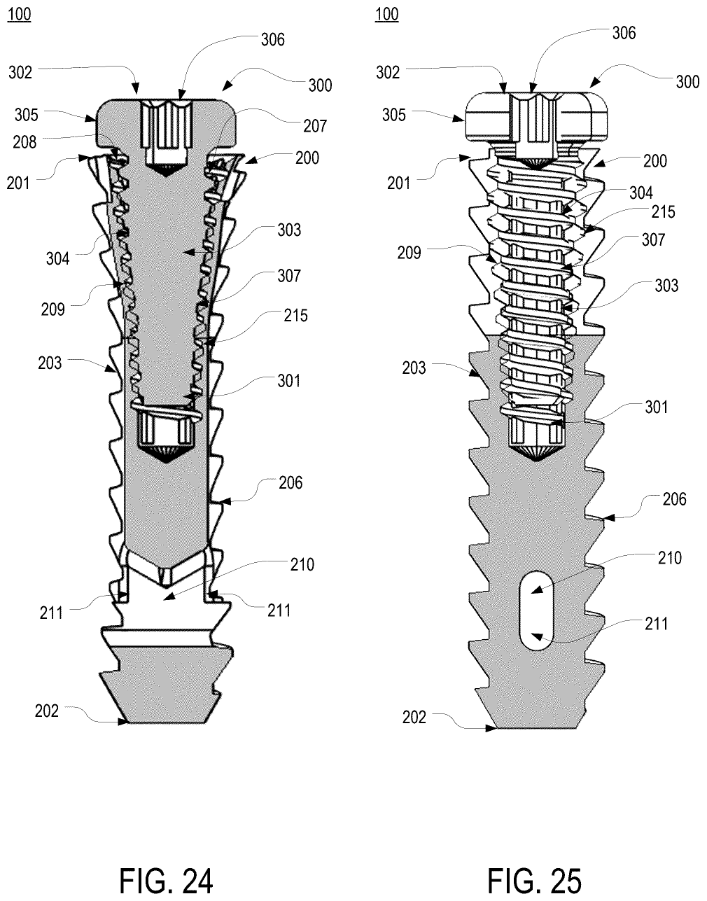

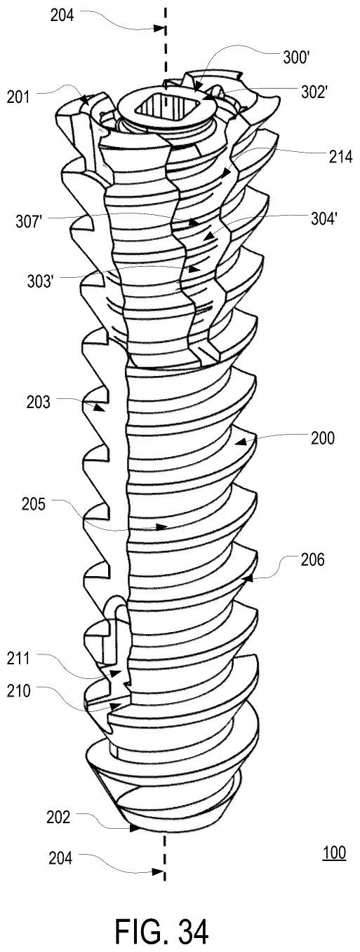

Referring back to FIGS. 7-9, 13, 15, 50-52 and 54-55, the body (203, 203′) of the anchor liner (200, 200′) may further comprise a cavity (207, 207′) bored through the body (203, 203′) extending from the proximal end (201, 201′) along the longitudinal axis (204, 204′). The cavity (207, 207′) may have an opening (208, 208′) at the proximal end (201, 201′) of the anchor liner (200, 200′). Referring to FIG. 24 and 33-34, the cavity (207, 207′) may be adapted to receive the inner locking component (300, 330′) through the opening (208, 208′) of the cavity (207, 207′). Referring further to FIGS. 8 and 55, a cross-sectional view of the anchor liner (200, 200′) is shown. The cavity (207, 207′) may have an inner wall (209, 209′). As depicted, the inner wall (209, 209′) of the cavity (207, 207′) may optionally have an internal thread (215, 215′). In some embodiments, such as the one depicted in FIG. 55, the internal thread (215′) of the inner wall (209′) may only extend partially along the cavity (207′) of the anchor liner (200′). In another embodiment, such as the one depicted in FIG. 8, the internal thread (215) of the inner wall (209) may extend along the entire cavity (207) of the anchor liner (200). In other embodiments, the inner wall (209, 209′) may optionally be smooth or textured (not shown). In further embodiments, the inner wall (209, 209′) may optionally comprise a rough surface or sharp teeth (not shown). The inner wall (209, 209′) of the cavity (207, 207′) may be adapted to engage with an outer surface (304, 304′) of the inner locking component (300, 300′) (see FIGS. 24-25 and 33).

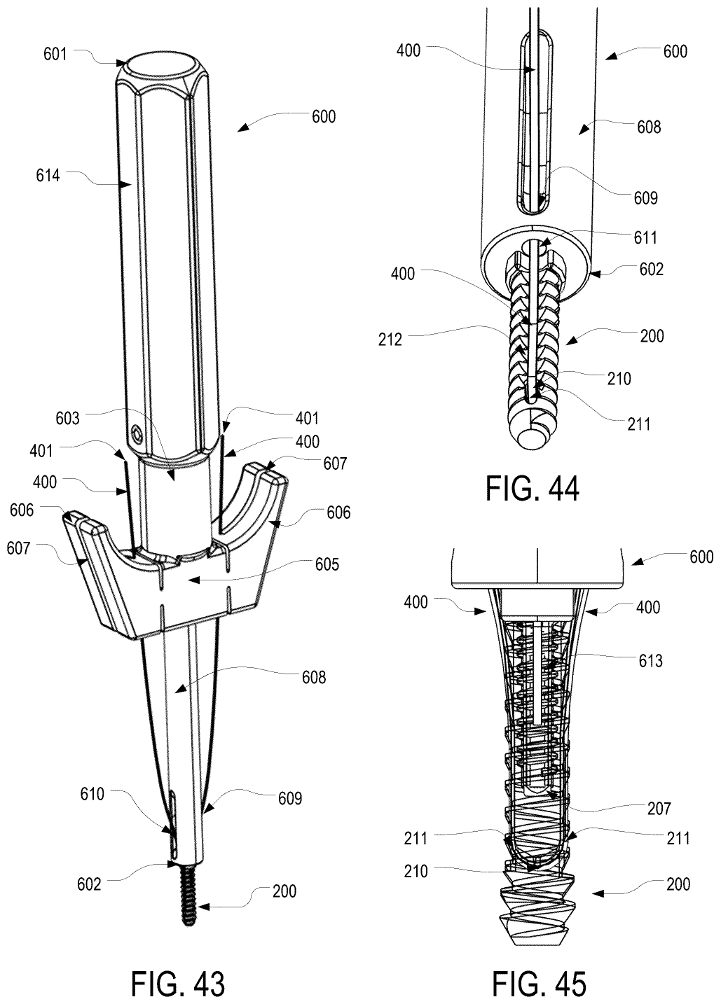

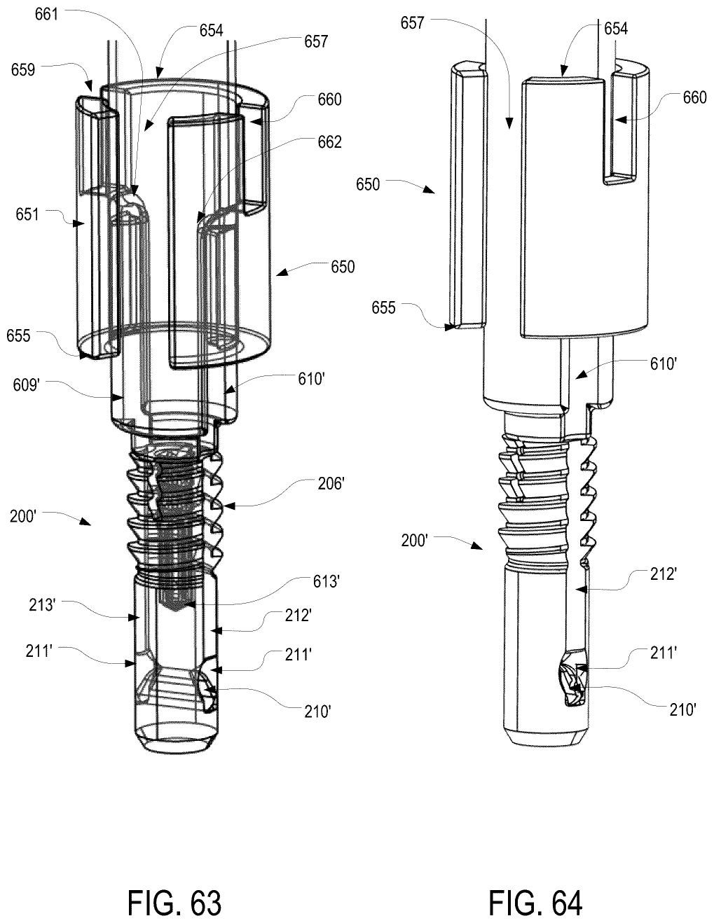

Referring next to FIGS. 43 through 48 and 61 through 63, in at least one embodiment, the cavity (207, 207′) of the anchor liner (200, 200′) may optionally be adapted to receive a driving tip (613, 613′) of an insertion tool (600, 600′) used to drive the anchor liner (200, 200′) into the bone (800). Referring to FIGS. 45 and 63, in such embodiments, the cavity (207, 207′) may be shaped to correspond with the shape of the driving tip (613, 613′) such that the driving tip (613, 613′) may engage the anchor liner (200, 200′) to impart the force to insert the anchor liner (200, 200′) into the bone (800). The anchor liner (200, 200′) and insertion tool (600, 600′) of FIGS. 45 and 63 are shown as transparent for illustrative purposes only.

Now referring back to FIGS. 7, 10-11, and 50-55, the body (203, 203′) of the anchor liner (200, 200′) may further comprise a suture hole (210, 210′) through the body (203, 203′) transverse to the longitudinal axis (204, 204′) and near the distal end (202, 202′) of the anchor liner (200, 200′). In embodiments of the anchor liner (200′) having a threaded section (216′) and a smooth section (217′), such as the one depicted in FIGS. 50 through 55, the smooth section (217′) may comprise the suture hole (210′) of the body (203′) and may extend from distal end (202′) to the threaded section (216′). The suture hole (210, 210′) may have openings (211, 211′) at either end of the suture hole (210, 210′). The suture hole (210, 210′) may be adapted to receive a suture (400) as shown in FIGS. 1, 5-6, 30, 33, 44-45 and 69-71. As shown in the cross-sectional side view of FIGS. 5 and 33, and the transparent view of FIG. 71, the suture hole (210, 210′) may receive a suture (400) through one opening (211, 211′) of the suture hole which then passes through the suture hole (210, 210′) and exits through an opposite opening (211, 211′) of the suture hole (210, 210′) located on an opposite side of the anchor liner (200, 200′) body (203, 203′).

Now referring again to FIGS. 7, 9-11, 13, 15, and 50-54, in some embodiments, such as the ones depicted, the body (203, 203′) of the anchor liner (200, 200′) may optionally have a first suture channel (212, 212′) and a second suture channel (213, 213′) parallel to the longitudinal axis (204, 204′) along opposite sides of the body (203, 203′). Referring further to FIGS. 7, 9 and 15, each suture channel (212, 213) may be recessed in the external thread (206) of the body (203) of the anchor liner (200). Referring to FIGS. 50-51 and 54, each suture channel (212′, 213′) may be recessed in the external thread (206′) of the threaded section (216′) and extend through the smooth section (217′). Now referring to FIGS. 7, 10-11, and 50-51, each suture channel (212, 212′, 213, 213′) may extend from the proximal end (201, 201′) of the anchor liner (200, 200′) to a respective opening (211, 211′) of the suture hole (210, 210′). Referring back to FIGS. 1, 4-6, 30, 33, 44-45 and 70-71, in at least one embodiment, the suture hole (210, 210′), the first suture channel (212, 212′) and the second suture channel (213, 213′) may be adapted to receive the suture (400). In such embodiments, the suture (400) may first be received by the first suture channel (212, 212′), followed by the suture hole (210, 210′) through entering one opening (211, 211′) of the suture hole (210, 210′), and then by the second suture channel (213, 213′) upon exiting the suture hole (210, 210′) through an opposite opening (211, 211′). In at least one embodiment, the second suture channel (213, 213′) may optionally receive the suture (400) first, followed by the suture hole (210, 210′) and then the first suture channel (212, 212′).

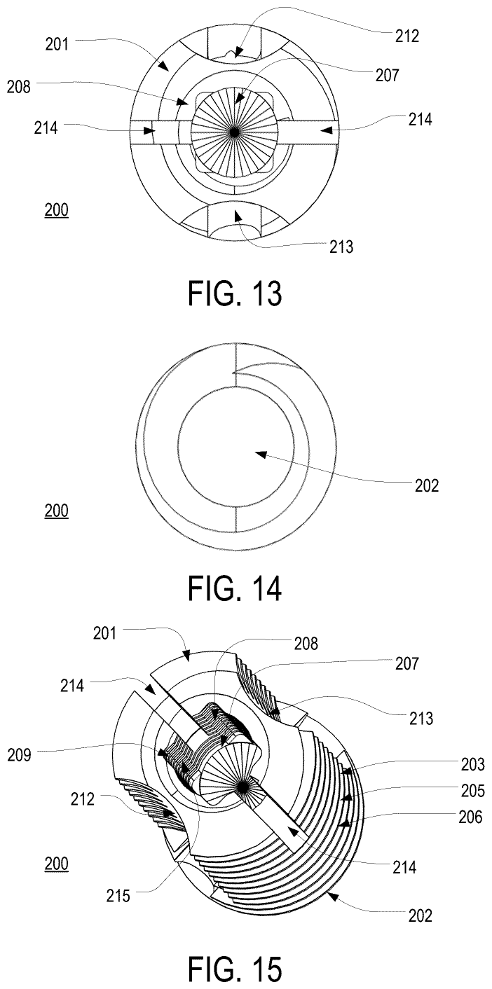

Referring next to FIGS. 7-9, 12-13, 15 and 50-51, the body (203, 203′) of the anchor liner (200, 200′) may further comprise one or more expansion areas (214, 214′) through a portion of the body (203, 203′) at the proximal end (201, 201′) of the anchor liner (200, 200′). Although the figures depict the anchor liner (200, 200′) with two expansion areas (214, 214′), the body (203, 203′) may have one or more expansion areas (214, 214′). In embodiments having more than one expansion area (214, 214′), the expansion areas (214, 214′) may optionally be the same or different sizes and the same or different shapes. Referring again to FIGS. 13, 15 and 50-52, in at least one embodiment having first (212, 212′) and second (213, 213′) suture channels, each of the one or more expansion areas (214, 214′) may be located radially between the first suture channel (212, 212′) and second suture channel (213, 213′).

Although the expansion areas (214, 214′) are depicted in the figures as slits, it should be understood that the one or more expansion areas (214, 214′) may optionally comprise, for example, slits, expansion channels, slices, weakened or thinned areas, elastic areas, gaps, or perforated areas.

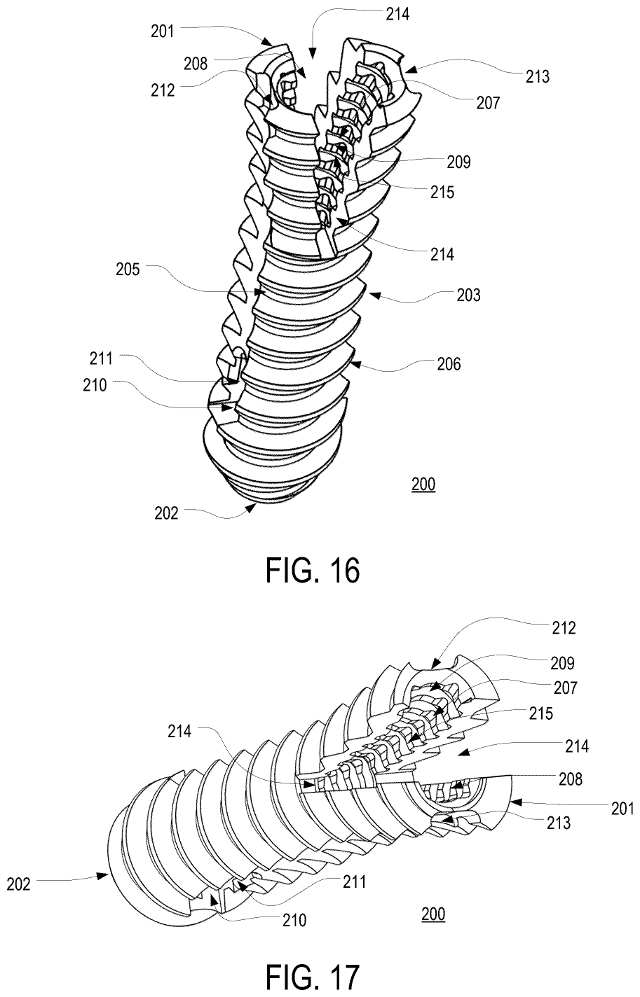

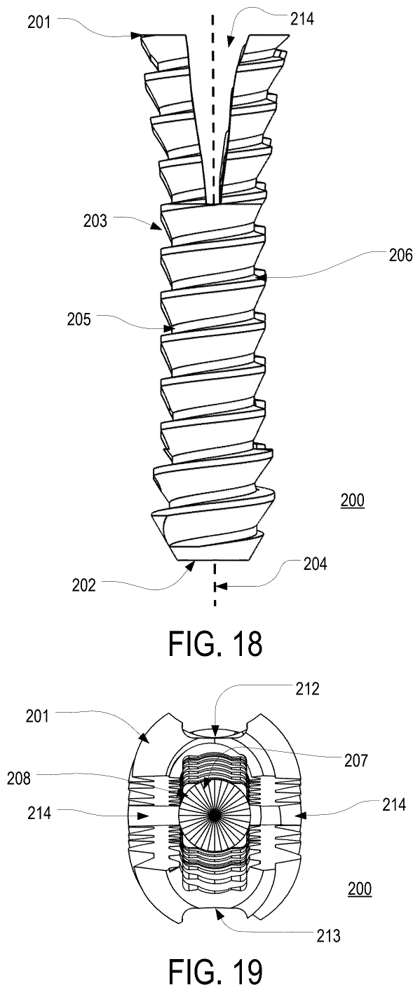

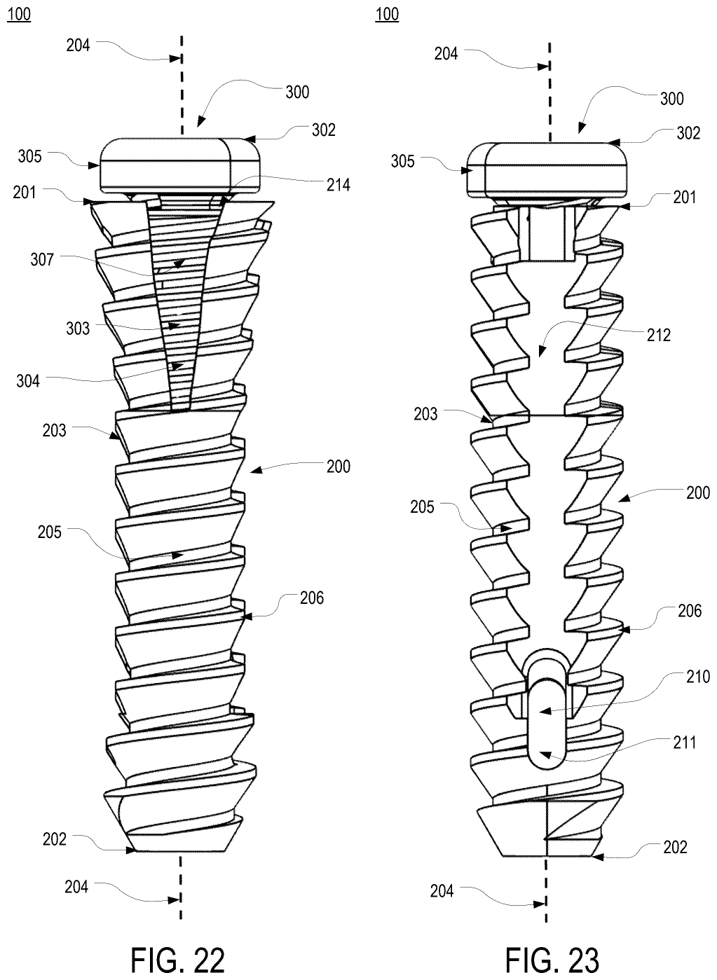

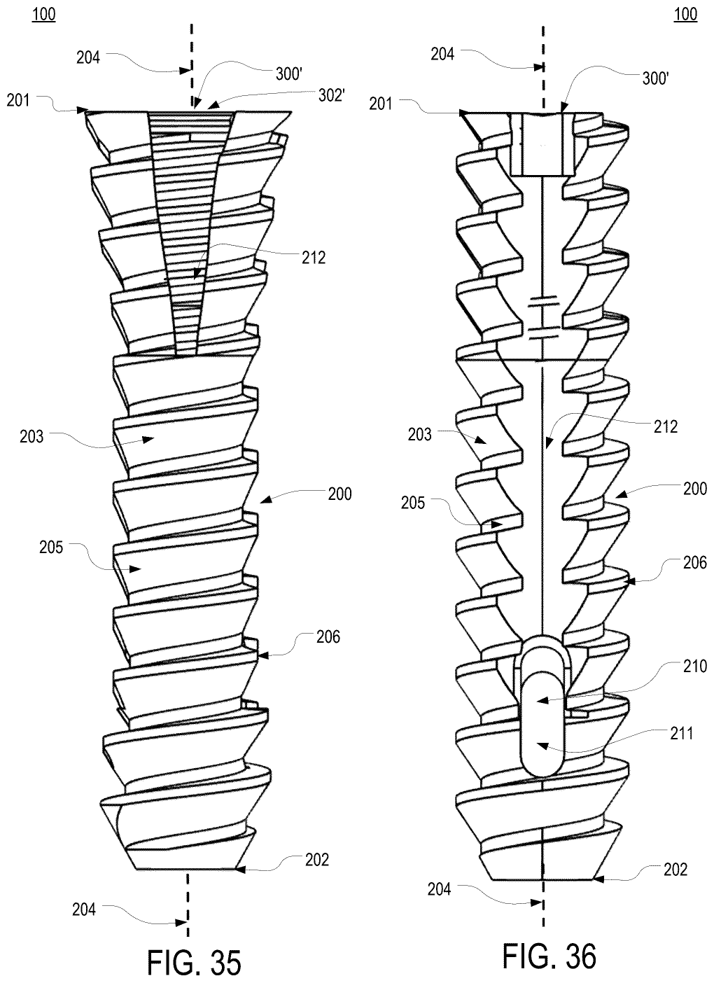

Referring now to FIGS. 22 and 35, in some embodiments, the one or more expansion areas (214) are adapted to expand the proximal end (201) of the anchor liner (200) when the anchor liner (200) receives the inner locking component (300, 300′). In further embodiments, the proximal end (201) of the anchor liner (200) may be adapted to progressively expand radially as the inner locking component (300, 300′) is inserted into the cavity (207) of the anchor liner (200). The anchor liner (200) may be further adapted to lock the suture (400) received by the system (100) between a bone and the anchor liner (200) as the proximal end (201) of the anchor liner (200) expands (see FIG. 49 suture anchor system (100) after inserted into the bone (800)). Referring to FIGS. 16-19, shown are various views of an anchor liner (200) after the proximal end (201) of the anchor liner (200) has expanded radially. As can be seen in the figures, the proximal end (201) of the anchor liner (200) may be adapted to expand such that it is wider than the rest of the body (203) and the distal end (202) of the anchor liner (200). The proximal end (201) of the anchor liner (200) may be adapted to wedge against a bone (not shown) as the proximal end (201) expands. The wedging effect locks the suture (400) between the anchor liner (200) and the bone (not shown).

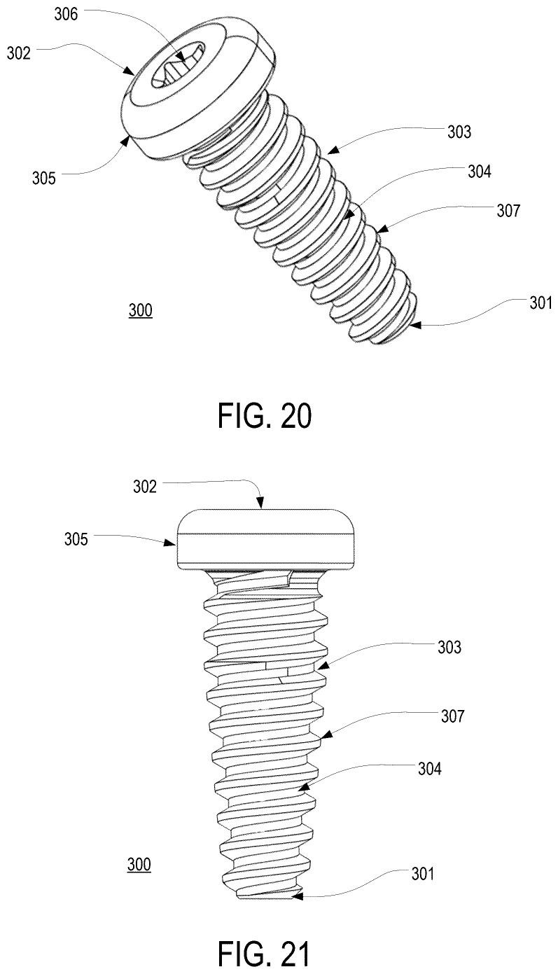

Referring next to FIGS. 20 and 21, shown are perspective and side views of an inner locking component (300) in accordance with one embodiment. The inner locking component (300) may be adapted to be received by the cavity (207) of the anchor liner (200), as shown in FIGS. 22-25. Referring back to FIGS. 20 and 21, the inner locking component (300) may comprise a leading end (301), a trailing end (302) and an elongated body (303) therebetween. In at least one embodiment, the trailing end (302) may optionally be adapted to receive a driving tool (not shown). In such embodiments, the trailing end (302) may comprise a socket (306) adapted to receive the driving tool. The body (303) of the inner locking component (300) may comprise an outer surface (304). In at least some embodiments, such as the ones depicted, the outer surface (304) may optionally comprise an external thread (307). In other embodiments, the outer surface (304) may optionally be smooth or textured (not shown). In some embodiments, the outer surface (304) may optionally comprise a rough surface or sharp teeth (not shown). In further embodiments, the outer surface (304) may have the ability to expand (not shown). Referring again to FIGS. 24 and 25, the outer surface (304) of the inner locking component (300) may be adapted to engage the inner wall (209) of the cavity (207) in the anchor liner (200).

In some embodiments, the outer surface (304) of the inner locking component (300) comprises an external thread (307) and the inner wall (209, 209′) of the cavity (207, 207′) of the anchor liner (200, 200′) comprises an internal thread (215, 215′). In such embodiments, the external thread (307) of the outer surface (304) may be adapted to engage, or mate with, the internal thread (215, 215′) of the inner wall (209, 209′).

In other embodiments, the outer surface (304) of the inner locking component (300) comprises an external thread (307) and the inner wall (209) of the cavity (207) of the anchor liner (200) is smooth or textured. In such embodiments, the external thread (307) of the outer surface (304) may be adapted to engage the inner wall (209) through the treads of the external thread (307) digging into the smooth or textured inner wall (209).

In further embodiments, the outer surface (304) of the inner locking component (300) is smooth or textured and the inner wall (209) of the cavity (207) of the anchor liner (200) comprises an internal thread (215). In such embodiments, the internal thread (215) of the inner wall (209) may be adapted to engage the outer surface (304) through the treads of the internal thread (215) digging into the smooth or textured outer surface (304) of the inner locking component (300).

In yet another embodiment, the outer surface (304) of the inner locking component (300) is textured and the inner wall (209) of the cavity (207) of the anchor liner (200) is textured. In such embodiments, the textured outer surface (304) may be adapted to engage the textured inner wall (209).

In at least one embodiment, the body (303) of the inner locking component (300) may optionally be substantially cylindrical. In further embodiments, the body (303) of the inner locking component (300) may optionally be tapered or frustoconical in shape with a diameter of the body (303) narrowing gradually from the trailing end (302) to the leading end (301) of the inner locking component (300). The tapered or frustoconical shape of the body (303) may be adapted to gradually expand the one or more expansion areas (214) of the anchor liner (200) as the inner locking component (300) is received by and inserted into the cavity (207) of the anchor liner (200), thereby progressively radially expanding the proximal end (201) of the anchor liner (200) (see, for example, FIG. 22 where the anchor liner (200) proximal end (201) is expanded after the anchor liner (200) has received the inner locking component (300)). The progressive expansion of the proximal end (201) of the anchor liner (200) is adapted to lock the suture (400) between the bone and the anchor liner (200) (not shown).

Referring to FIGS. 21 and 22, in some embodiments, the trailing end (302) of the inner locking component (300) may optionally further comprise a head (305). In such embodiments, the head (305) may be adapted to receive the driving tool. The head (305) may optionally have a socket (306) adapted to receive the driving tool. When the inner locking component (300) is received by the cavity (207) of the anchor liner (200), the head (305) may optionally have a diameter wider than a diameter of the cavity (207) such that the head (305) is adapted to prevent the inner locking component (300) from completely entering the cavity (207) of the anchor liner (200). In some embodiments the head (305) may be further adapted to lock the suture (400) between the head (305) and the anchor liner (200) or the head (305) and the bone (800).

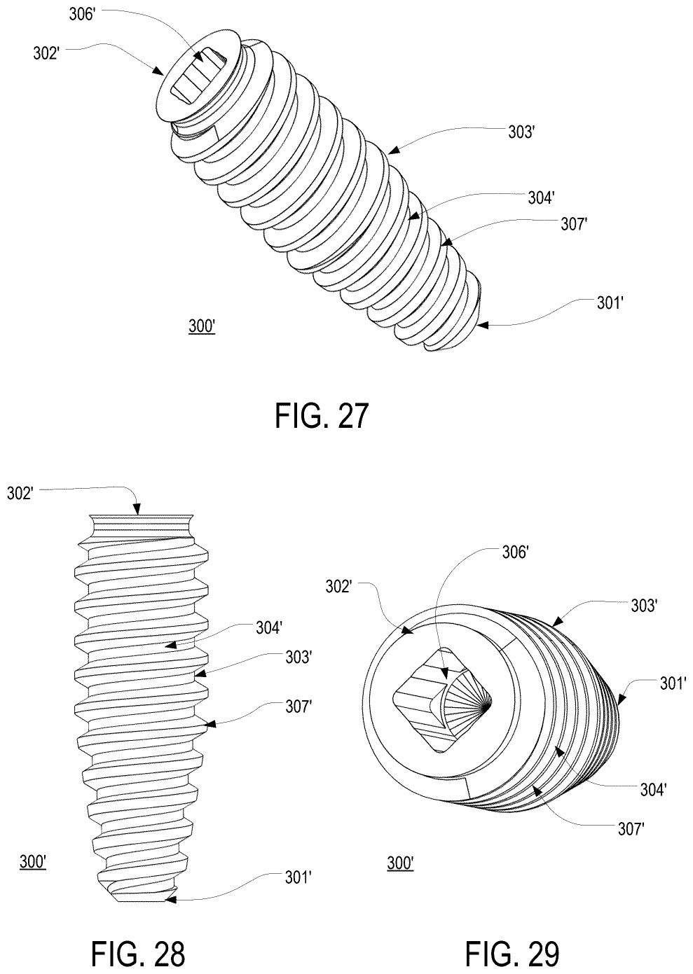

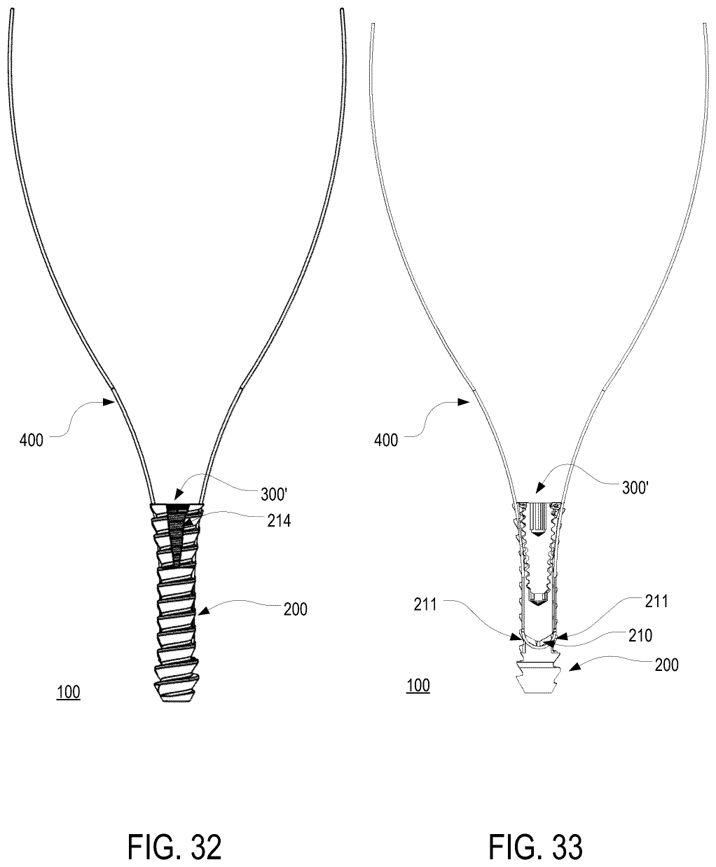

Now referring to FIGS. 27 through 29, in at least one embodiment, the suture anchor system (100) may optionally comprise a headless inner locking component (300′). The trailing end (302′) of the inner locking component (300′) may optionally be headless. In such embodiments, the trailing end (302′) may be adapted to receive the driving tool. The trailing end (302′) may optionally have a socket (306′) adapted to receive the driving tool. Referring to FIGS. 30, 32-36 and 49, when the headless embodiment of the inner locking component (300′) is received by and fully inserted into the cavity (207) of the anchor liner (200), the trailing end (302′) of the inner locking component (300′) may optionally sit flush with the opening (208) of the cavity (207) at the proximal end (201) of the anchor liner (200).

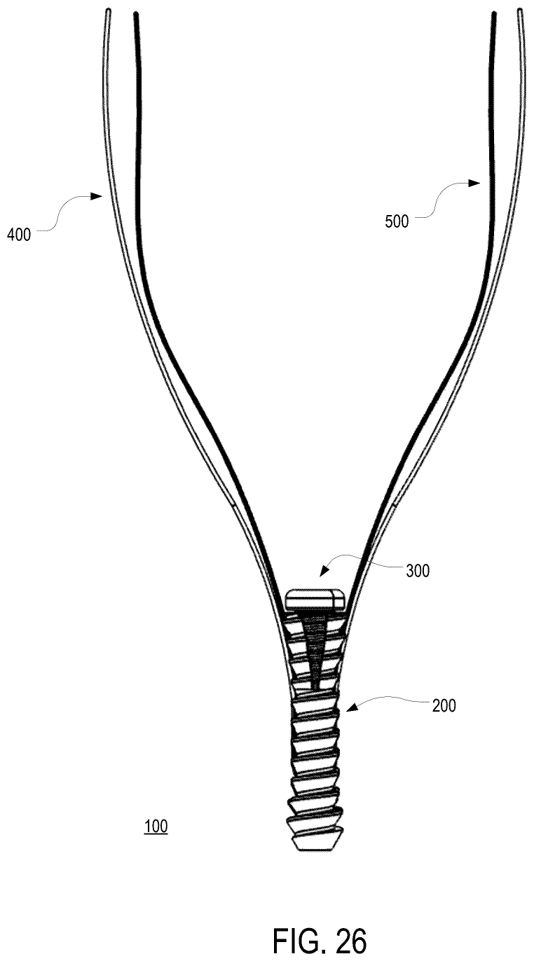

Referring to FIG. 26, in at least one embodiment, the suture anchor system (100) may be adapted to receive both a suture (400) and a suture tape (500). In such embodiments, the suture hole (210), the first suture channel (212), and the second suture channel (213) may be adapted to receive the suture (400) and the suture tape (500).

In some embodiments, the suture anchor system (100) may be absorbable, non-absorbable, or biocomposite. Non-absorbable embodiments may be made of, for example, a biocompatible metallic material (e.g., Titanium) or polyether ether ketone (PEEK). Absorbable embodiments may be made of a biodegradable material, such as, for example, Poly-L-lactic acid (PLLA) or poly-L-co-D, L-lactic acid (PLDLA). Biocomposite embodiments may comprise, for example, poly(L-lactide-co-D, L-lactide) (PLDLA) or beta tricalcium phosphate (β-TCP).

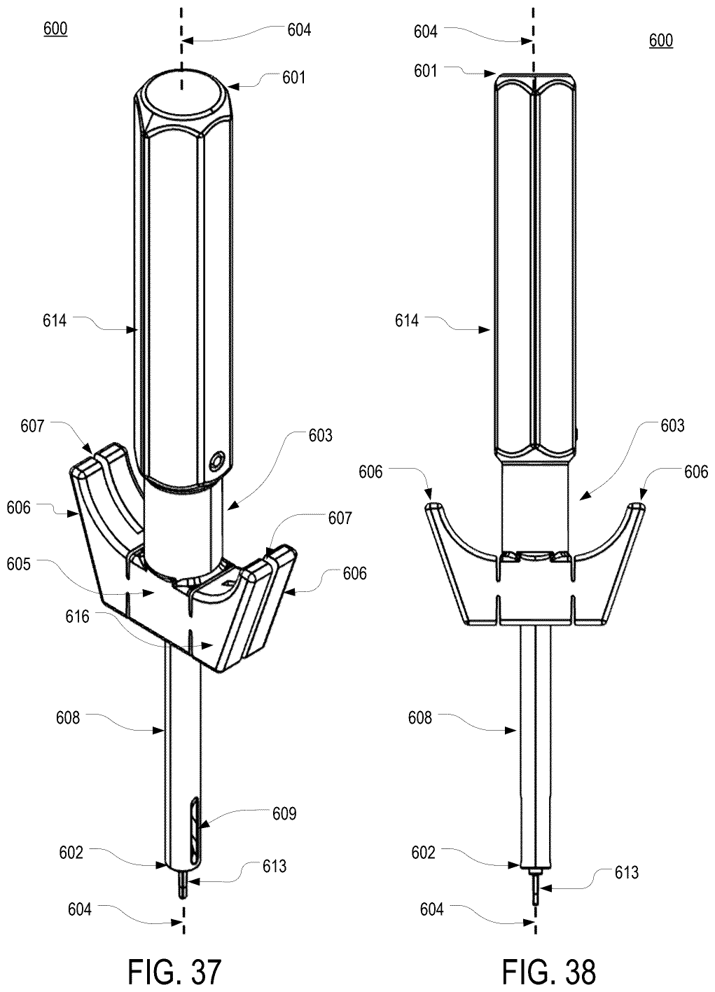

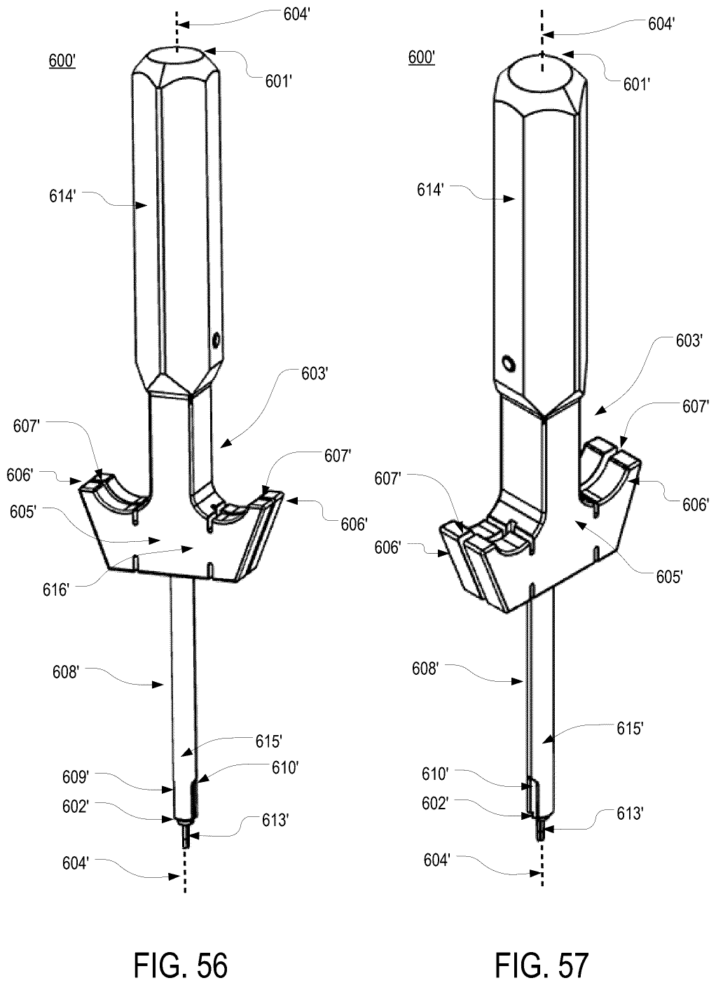

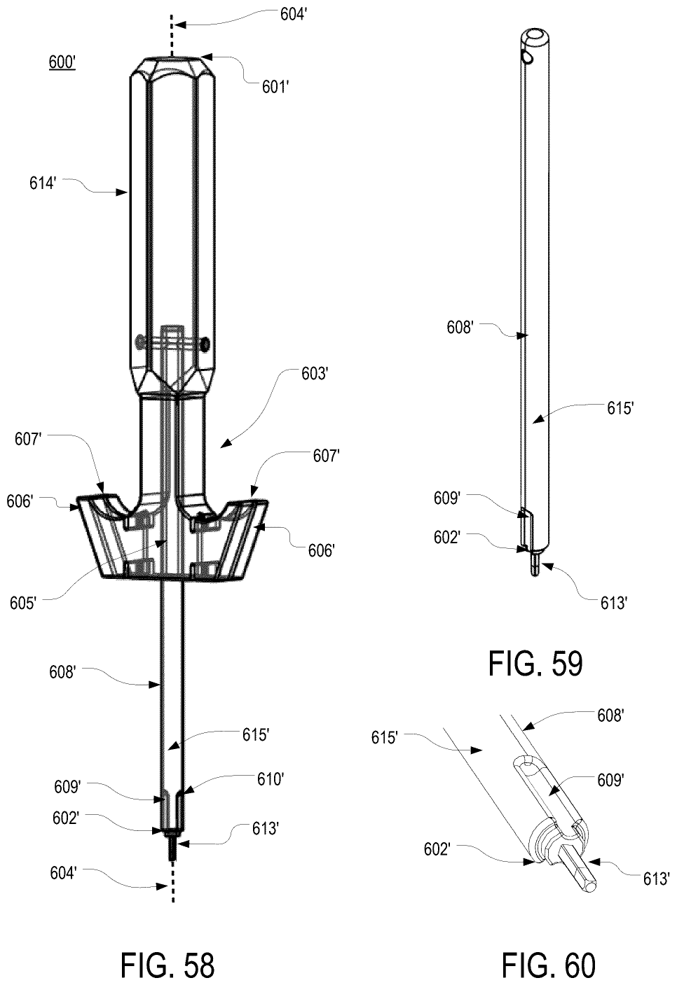

Next, referring to FIGS. 37 through 42, shown are various views of an insertion tool (600) adapted for inserting the anchor liner (200) of the suture anchor system (100) into a bone (800) and tensioning the suture (400) therein. Referring to FIGS. 56-64, shown are various views of an insertion tool (600′) in accordance with another embodiment. The insertion tool (600, 600′) may comprise a proximal end (601, 601′), a distal end (602, 602′) and a body (603, 603′) with a longitudinal axis (604, 604′) therebetween.

The body (603, 603′) may optionally comprise a handle (605, 605′) and an elongated shaft (608, 608′) extending from the handle (605, 605′) to the distal end (602, 603′) of the insertion tool (600, 600′). Referring to FIGS. 37 and 56, the handle may optionally further comprise a suture retention mechanism (616, 616′). Referring to FIGS. 37-38, 41, 56-58 and 61-62, in at least some embodiments, such as the ones depicted, the suture retention mechanism (616, 616′) of the handle (605, 605′) may optionally further comprise one or more arms (606, 606′). Although the embodiment depicted throughout the figures includes two arms (606, 606′), this is not the only possible embodiment of the insertion tool (600, 600′). The arms (606, 606′) may optionally be made from a plastic material. In at least some embodiments, the one or more arms (606, 606′) may optionally be rotatably engaged with the body (603, 603′). Each arm (606, 606′) may further comprise a slit or cleat (607, 607′) extending substantially transverse to the longitudinal axis (604, 604′) of the insertion tool (600, 600′). Each slit (607, 607′) on each arm (606, 606′) may be adapted to receive an end (401) of a suture (400) (see FIG. 43, 46-47 and 69-71) for tensioning. Each slit (607, 607′) may be further adapted to temporarily secure the suture (400) when the insertion tool (600, 600′) is used to insert or rotate the anchor liner (200, 200′) of the suture anchor system (100).

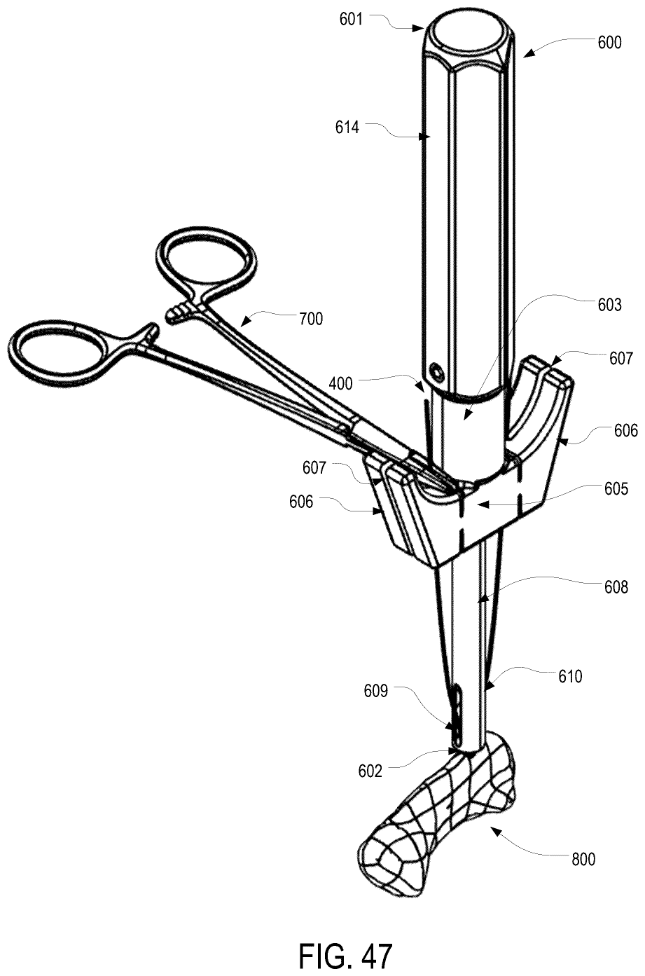

In at least some embodiments, such as the ones depicted, the shape of the one or more arms (606, 606′) may form one or more winged arms. In such embodiments, each winged arm (606, 606′) may curve proximally upward, forming a valley between an edge of the winged arm (606, 606′) and the body (603, 603′) of the insertion tool (600, 600′). Referring to FIG. 47, each of the one or more arms (606, 606′) may be adapted to receive an instrument (700) for tensioning the suture (400). In embodiments having one or more winged arms (606, 606′), each of the winged arms (606, 606′) may receive the instrument (700) for tensioning the suture (400) such that the instrument (700) may rest in the valley of the winged arm (606, 606′) when tensioning the suture (400). The instrument (700) may be a surgical instrument such as a hemostat, needle driver, clamp, forceps, or any other instrument capable of holding, grasping, or clamping an end (401) of the suture (400).

Referring to FIGS. 37, 39-40, 42-44, 46 and 56-60, the shaft (608, 608′) of the body (603, 603′) of the insertion tool (600, 600′) may be substantially cylindrical in one embodiment. The shaft (608, 608′) may optionally comprise at least one channel on the shaft (608, 608′). In some embodiments, such as the one depicted throughout FIGS. 37-44, the at least one channel (609, 610) may be bored through the shaft (608) forming a tunnel. In other embodiments, such as the one depicted throughout FIGS. 56-60, the at least one channel (609′, 610′) may be recessed along an exterior (615′) of the shaft (608′). The at least one channel may be located anywhere along the length of the shaft (608, 608′). In at least one embodiment, the at least one channel may optionally be located on the shaft (608, 608′) towards the distal end (602, 602′) of the insertion tool (600, 600′). In other embodiments, the at least one channel may optionally be located on the shaft (608, 608′) towards the handle (not shown).

In the embodiment depicted throughout FIGS. 37-44, the shaft (608) comprises both a first channel (609) and a second channel (610), each channel extending through the shaft (608) from opposite sides of the shaft (608) to respective first (611) and second (612) suture holes, and forming a tunnel therebetween. In at least some embodiments, such as the one depicted in FIGS. 37-44, the first (611) and second (612) suture holes may optionally be located at the distal end (602) of the insertion tool (600). The first channel (609) may extend from a side of the shaft (608) to the corresponding first suture hole (611), and the second channel (610) may extend from an opposite side of the shaft (608) to the corresponding second suture hole (612).

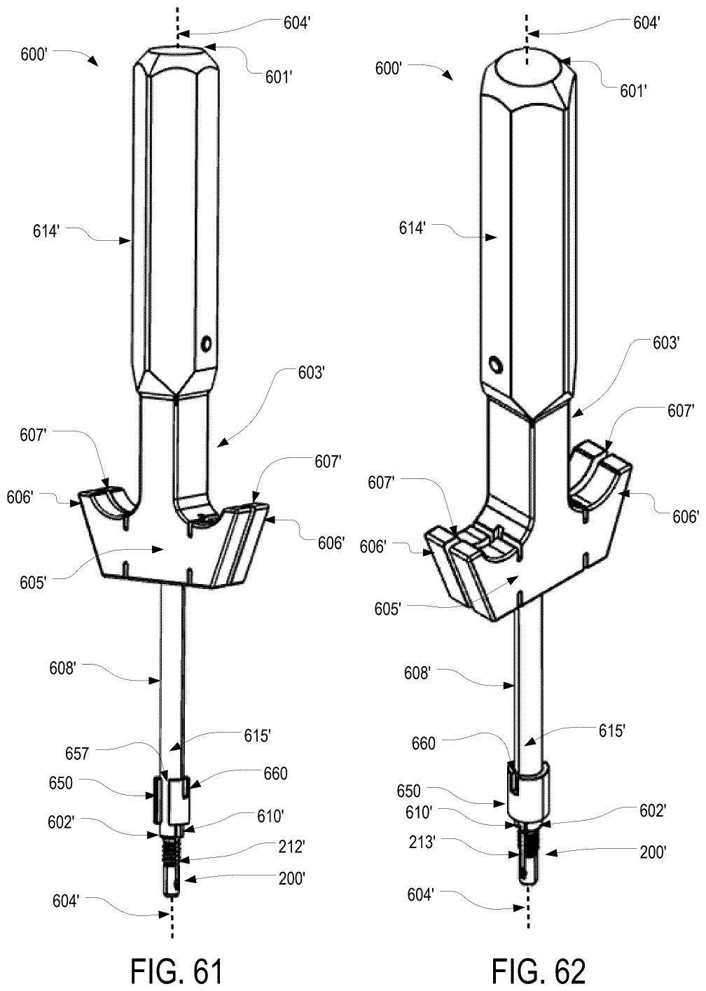

In the embodiment depicted throughout FIGS. 56-60, the shaft (608′) of the body (603′) comprises a first channel (609′) and a second channel (610′) recessed along the exterior (615′) of the shaft (608′), the first (609′) and second (610′) channels being on opposite sides of the shaft (608′). In the embodiment of FIGS. 56-60, the first (609′) and second (610′) channels each extend from the distal end (602′) of the insertion tool and are adapted to align with respective first (212, 212′) and second (213, 213′) suture channels of the anchor liner (200, 200′) (see, for example, FIGS. 63 and 64). Referring to FIGS. 61-62 and 69-71, in at least some embodiments, the insertion tool (600′) may optionally comprise a detachable suture alignment clip (650).

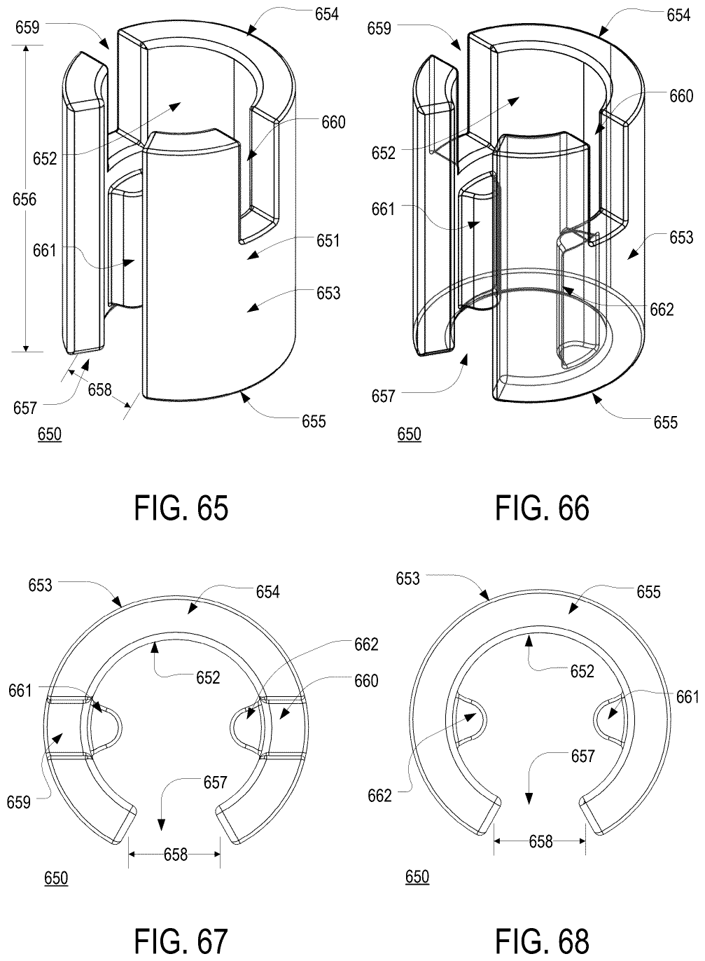

Referring to FIGS. 65-68, shown are various views of a suture alignment clip (650) in accordance with one embodiment. The suture alignment clip (650) may comprise a substantially cylindrical hollow body (651) having a proximal end (654), a distal end (655) and a length (656) therebetween, an opening slit (657), first and second side slits (659, 660), and first and second protrusions (661, 662). The hollow body has an interior (652) and exterior (653) side. It should be understood that although the suture alignment clip (650) is depicted in the figures as cylindrical, other shapes are possible. The shape of the body (651) of the suture alignment clip (650) is such that its interior (652) shape corresponds with the shape of the shaft (608′) of the insertion tool (600′). The opening slit (657) may extend from the proximal end (654) to the distal end (655) for the entire length (656) of the body (651). The opening slit (657) has an opening width (658) that is adapted to allow the suture alignment clip (650) to attach to and detach from the shaft (608′) at the distal end (602, 602′) of the insertion tool (600, 600′). The opening slit (657) allows for easy installation and removal of the suture alignment clip (650) to and from the shaft (608′) of the insertion tool (600′). The material of the suture alignment clip (650) may be a flexible material. The first (659) and second (660) side slits are located on opposite sides of the body (651) and extend from the proximal end (654) partially along the length (656) of the body (651). The first (661) and second (662) protrusions are located on the interior (652) of the body (651) and extend from the distal end (655) partially along the length (656) of the body, optionally to the respective first (659) and second (660) side slits. The first protrusion (661) on the interior (652) aligns with the first side slit (659) and the second protrusion (662) on the interior (652) aligns with the second side slit (660). Referring to FIGS. 63 and 71, the first (661) and second (662) protrusions are adapted to fit into and align with respective first (609′) and second (610′) channels on the shaft (608′) of the insertion tool (600′). The first (661) and second (662) protrusions may be adapted to secure the suture alignment clip (650) to the insertion tool (600′).

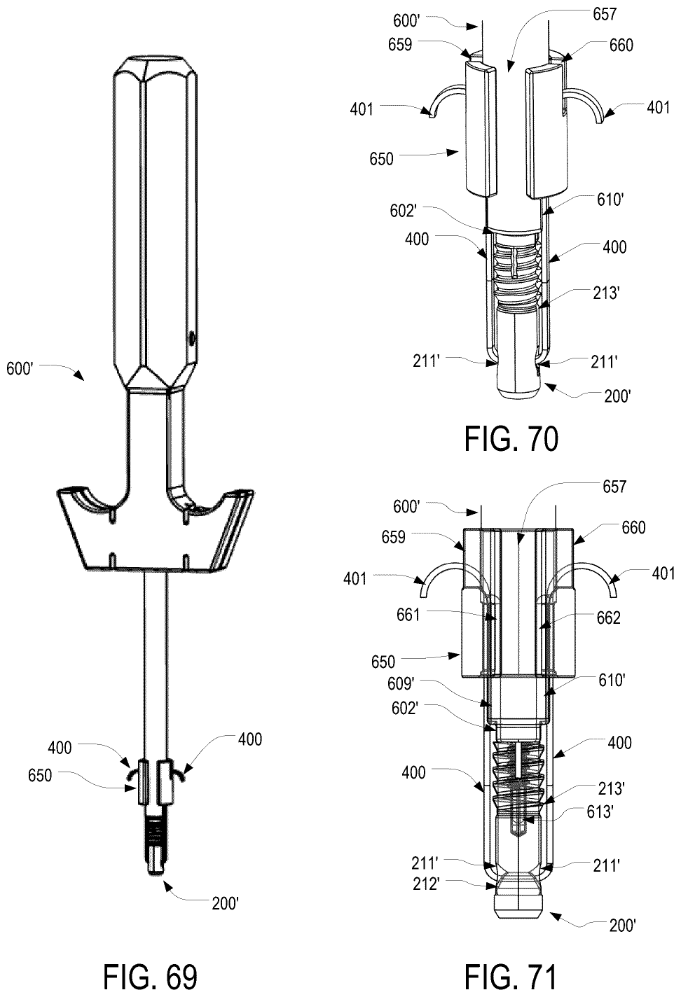

Referring further to FIGS. 63 and 71, in embodiments having a suture alignment clip (650), the first protrusion (661) is adapted to be received by and mate with the first channel (609′) on the shaft (608′) of the insertion tool (600′) and compress a suture (400) received by the first channel (609′) of the shaft (608′). The second protrusion (662) is adapted to be received by and mate with the second channel (610′) of the insertion tool (600′) and compress a suture (400) received by the second channel (610′) of the shaft (608′). The compression of the first (661) and second (662) protrusions against the respective first (609′) and second (610′) channels keeps the suture (400) aligned and vertical with respect to the corresponding first (212, 212′) and second (213, 213′) suture channels of the anchor liner (200, 200′) when the insertion tool (600′) is received by the anchor liner (200, 200′). Referring to FIGS. 69-71, the first side slit (659) is adapted to allow an end (401) of the suture (400) to exit the suture alignment clip (650), and second side slit (660) is adapted to allow the another end (401) of the suture (400) to exit the suture alignment clip (650) when the suture alignment clip (650) is attached to the distal end (602′) of the insertion tool (600′). The first (659) and second (660) side slits allow for a user to tension the suture (400).

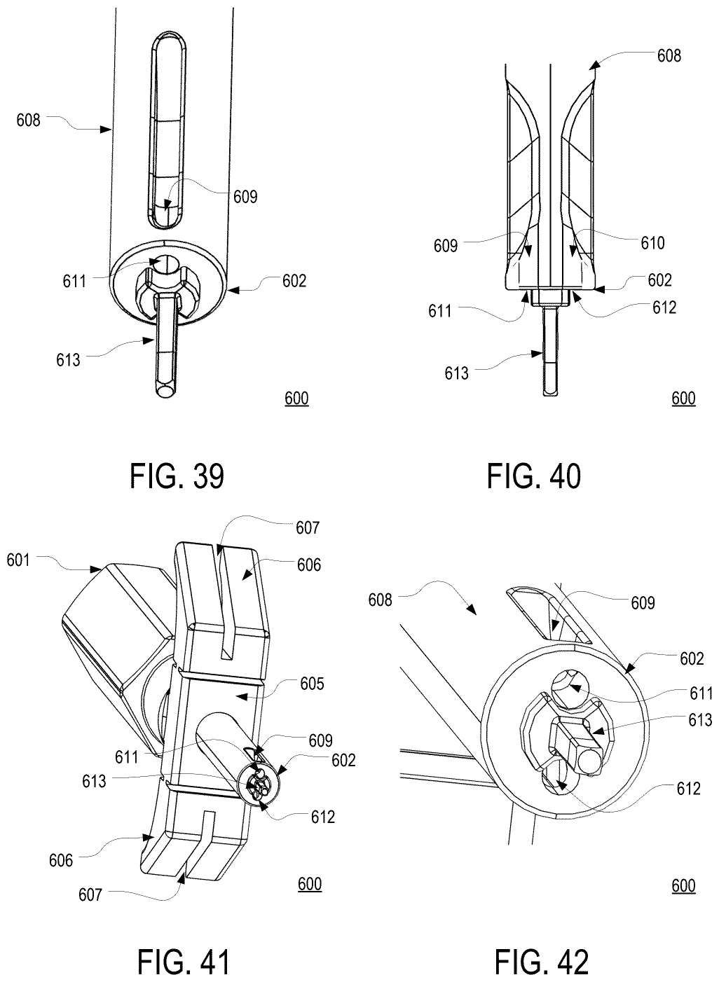

Referring to FIGS. 39-42, 45 and 56-60, the distal end (602, 602′) of the insertion tool (600, 600′) may further comprise a driving tip (613, 613′). The driving tip (613) may extend distally outward from the distal end (602) of the insertion tool (600). In at least one embodiment, such as the one depicted in FIGS. 39-42 and 45, the driving tip (613) may optionally be located between the first suture hole (611) and the second suture hole (612). Referring further to FIGS. 45, 63-64 and 71, the distal end (613, 613′) may be adapted to be received by the anchor liner (200, 200′) of the suture anchor system (100). Optionally, the driving tip (613, 613′) may be received by the cavity (207, 207′) of the anchor liner (200, 200′). It should be noted that the anchor liner (200, 200′) of FIGS. 45, 63 and 71 is shown as transparent for illustrative purposes only in order to show the interaction of the driving tip (613, 613′) with the anchor liner (200, 200′).

The driving tip (613, 613′) may be adapted to engage the cavity (207, 207′) of the anchor liner (200, 200′). The driving tip (613, 613′) may be further adapted to drive the anchor liner (200, 200′) into a bone (800). The driving tip (613, 613′) may optionally be shaped to interact with and engage the cavity (207, 207′) of the anchor liner (200, 200′) such that the driving tip (613, 613′) may be adapted to drive the anchor liner (200, 200′) into the bone (800) through rotation and/or longitudinal force.

Referring to FIGS. 63-64, the first (609′) and second (610′) channels on the shaft (608′) of the insertion tool (600′) may be adapted to align with the first (212, 212′) and second (213 213′) suture channels, respectively, of the anchor liner (200, 200′) when the insertion tool (600′) is received by the anchor liner (200, 200′). In embodiments having a suture alignment clip (650), the first (659) and second (660) protrusions on the interior (652) of the suture alignment clip (650) are positioned to align with the first (609′) and second (610′) channels, respectively, of the shaft (608′) of the insertion tool (600′) and the first (212, 212′) and second (213, 213′) suture channels of the anchor liner (200, 200′) when the insertion tool (600′) is received by the anchor liner (200, 200′) such that the suture alignment clip (650) may slide or glide over and seamlessly across the suture channels (212, 212′, 213, 213′) of the anchor liner and the channels (609′, 610′) of the shaft (608′). Together, the alignment of the shaft's (608′) first and second channels (609′, 610′), the anchor liner's (200, 200′) first and second suture channels (212, 212′, 213, 213′) and the suture alignment clip's (650) first and second protrusions (659, 660) maintain alignment of a suture (400) received by the anchor liner (200, 200′) when inserting the anchor liner (200, 200′) into a bone (800) using the insertion tool (600′).

Referring back to FIGS. 43-45, shown is an embodiment of the insertion tool (600) having a first suture hole (611), second suture hole (612), first channel (609), and second channel (610). In such embodiments, the first suture hole (611) leading to the first channel (609) of the insertion tool (600) may be adapted to align with the respective first suture channel (212) of the anchor liner (200, 200′) (see anchor liner (200) of FIG. 15, (200′) of FIG. 54). The second suture hole (612) leading to the second channel (610) of the insertion tool (600) may be adapted to align with the respective second suture channel (213, 213′) of the anchor liner (200, 200′) (see anchor liner (200) of FIG. 15, (200′) of FIG. 54). The first suture hole (611) and the first channel (609) of the insertion tool (600) may be adapted to receive an end (401) of the suture (400). The second suture hole (612) and second channel (610) of the insertion tool (600) may be adapted to receive another end (401) of the suture (400). Together, the first (609) and second (610) channels and the respective first (611) and second (612) suture holes are adapted to maintain alignment of the suture (400) when inserting the anchor liner (200, 200′) into the bone (800) after the anchor liner (200, 200′) has received the suture (400).

Referring to FIGS. 37-38 and 56-58, the proximal end (601, 601′) of the insertion tool (600, 600′) may optionally comprise a gripping member (614, 614′). The gripping member (613, 613′) may optionally be of a plastic or rubber material. The shaft (608, 608′) and driving tip (613, 613′) may optionally be made from metal.

In at least one further embodiment, the insertion tool (600, 600′) may be adapted to be used in conjunction with a hammer or a mallet (not shown) to drive the anchor liner (200, 200′) into the bone without screwing the anchor liner (200, 200′). In such embodiments, the proximal end (601, 601′) of the insertion tool (600, 600′) may further comprise a surface adapted to receive blows or an impact force exerted by the hammer or the mallet (not shown). The impact of the blows from the hammer or mallet to the surface provides the longitudinal force for the driving tip (613, 613′) to drive the anchor liner (200, 200′) into the bone (800). It should be understood that the surface may be adapted to receive impact from other tools capable of providing longitudinal force to drive the anchor liner (200) into the bone (800).

### Method of Use

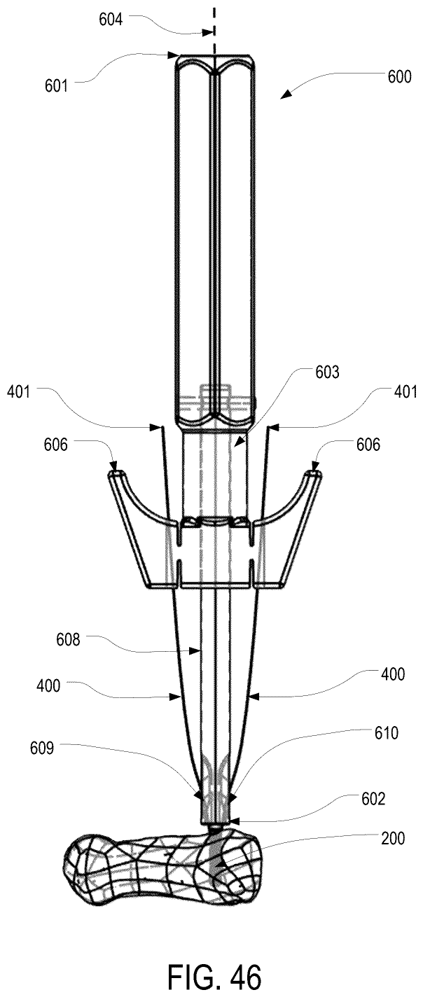

In operation, the following is a listing of the steps typically taken to utilize the disclosed suture anchor system (100) to secure a suture (400) to a bone:
1. Inserting one end (401) of a suture (400), if applicable, through an optional first suture channel (212, 212′) of an anchor liner (200, 200′) (see FIGS. 7-15 and 50-55), into one opening (211, 211′) of a suture hole (210, 210′) and through the suture hole (210, 210′) to exit an opposite opening (211, 211′) of the suture hole (210, 210′), and, if applicable, through an optional second suture channel (213, 213′) of the anchor liner. In embodiments without first (212, 212′) and second (213, 213′) suture channels, the one end of the suture (400) may be inserted into the one opening (211, 211′) of the suture hole (210, 210′), through the suture hole (210, 210′) and exit to an opposite opening (211, 211′) of the suture hole (210, 210′).
2. Inserting the anchor liner (200, 200′) into the bone. In some embodiments, an external thread (206, 206′) of the anchor liner (200) body (203) may engage the bone through rotation. In other embodiments, the external thread (206, 206′) may engage the bone through longitudinal force. In further embodiments, the external thread (206, 206′) may engage the bone through either rotation or longitudinal force.
   a. In at least some embodiments, an insertion tool (600, 600′) (see FIGS. 37-42 and FIGS. 56-62) may optionally be used to insert and drive the anchor liner (200, 200′) into the bone (800) (see FIG. 46-48). In methods using the insertion tool (600, 600′), the following is a listing of the steps typically taken to utilize the disclosed insertion tool (600, 600′) to insert the anchor liner (200) into the bone and tension the suture (400) therein:
      i. Inserting a driving tip (613, 613′) located at a distal end (602, 602′) of the insertion tool (600, 600′) into a cavity (207, 207′) of the anchor liner (200) (see FIGS. 43-45 and 61-64).
      ii. Optionally, in embodiments of the insertion tool (600) having a first (609) and second (610) channel bored through a shaft of the insertion tool and leading to first (611) and second (612) suture holes (see FIGS. 43-45), inserting the end (401) of the suture (400) that extends from the first suture channel (212) of the anchor liner (200) into a first suture hole (611) at the distal end (602) of the insertion tool (600) and through a first channel (609) of a shaft (608) of the insertion tool (600), the first channel (609) extending from the first suture hole (611) to a side of the shaft (608) (see FIG. 44). Alternatively, in embodiments of the insertion tool (600′) having first (609′) and second (610′) channels recessed in a shaft (608′) of the insertion tool (600′) and a suture alignment clip (650) adapted to attach to the shaft (608′) (see FIGS. 61-64), aligning the end (401) of the suture (400) that extends from the first suture channel (212, 212′) of the anchor liner (200, 200′) with the first channel (609′) on the shaft (608′) of the insertion tool (600′). In at least some embodiments, the insertion tool may not have any suture channels or suture holes. In such embodiments, steps 2(a)(ii) and (iii) may be skipped.
      iii. Optionally, in embodiments of the insertion tool (600) having a first (609) and second (610) channel bored through a shaft of the insertion tool and leading to first (611) and second (612) suture holes (see FIGS. 43-45), inserting the end (401) of the suture (400) that extends from the second suture channel (213) of the anchor liner (200) into a second suture hole (612) at the distal end (602) of the insertion tool (600) and through a second channel (610) of the shaft (608) of the insertion tool (600), the second channel (610) extending from the second suture hole (612) to a side of the shaft (608) opposite to the first channel (609). The first suture hole (611) and the second suture hole (612) are adapted to maintain alignment of the suture (400) when the anchor liner (200) is inserted into the bone (800) (see FIGS. 43 and 48). Alternatively, in embodiments of the insertion tool (600′) having first (609′) and second (610′) channels recessed in the shaft (608′) of the insertion tool (600′) and a suture alignment clip (650) adapted to attach to the shaft (608′) (see FIGS. 61-64), aligning the end (401) of the suture (400) that extends from the second suture channel (213, 213′) of the anchor liner (200, 200′) with the second channel (610′) on the shaft (608′) of the insertion tool (600′), and optionally sliding the suture alignment clip (650) over the optional first and second channels (212, 212′, 213, 213′) of the anchor liner and the first and second channels (609′, 610′) of the shaft (608′) of the insertion tool (600′) to secure the suture (400) in place (see FIGS. 69-71). This step may optionally be skipped in embodiments where one end (401) of the suture (400) is already engaged and secured by, for example, another suture anchor system (100) or a solid bodied anchor.
      iv. Optionally, inserting at least one end (401) of the suture (400) that was optionally passed through either the first channel (609, 609′) or the second channel (610, 610′) through a suture retention mechanism (616, 616′) of a handle (605, 605′) of the insertion tool (600, 600′). In embodiments where the suture retention mechanism (616, 616′) further comprises one or more arms (606, 606′) extending from the handle (605, 605′) of the insertion tool (600, 600′), this step may comprise inserting at least one end (401) of the suture (400) that was optionally passed through either the first channel (609, 609′) or the second channel (610, 610′) through a respective slit (607, 607′) on a respective one of two arms (606, 606′) (see, for e.g., FIG. 43). The slits (607, 607′) may extend in a direction transverse to a longitudinal axis (604, 604′) of the insertion tool (600, 600′). Each slit (607, 607′) may be adapted to temporarily secure the suture (400) when the insertion tool (600, 600′) is used to insert or rotate the anchor liner (200, 200′) in the bone (800). In embodiments where Step 2(a)(iii) is skipped and only one end (401) of the suture (400) is optionally passed through a respective first (609, 609′) or second (610, 610′) channel, only one end (401) of the suture (400) is received by the suture retention mechanism (616, 616′), or in some embodiments, a slit (607, 607′) on a respective arm (606, 606′) of the suture retention mechanism (616, 616′). In embodiments where Step 2(a)(iii) is included and each end (401) of the suture (400) is passed through a respective first channel (609, 609′) and second channel (610, 610′), each end (401) of the suture (400) is received by the suture retention mechanism (616, 616′), or in some embodiments, a respective slit (607, 607′) on a respective arm (606, 606′) of the suture retention mechanism (616, 616′).
      v. Inserting the anchor liner (200, 200′) into the bone using the driving tip (613, 613′) of the insertion tool (600, 600′) to provide rotation and/or longitudinal force (optionally with a hammer or mallet) to the anchor liner (200, 200′) such that the anchor liner (200, 200′) engages the bone (see FIG. 46; anchor liner (200, 200′) inserted into bone (800)). The handle (605, 605′) of the insertion tool (600, 600′) may optionally be used to provide rotation and/or longitudinal force to the anchor liner (200, 200′). A proximal end (601, 601′) of the insertion tool (600, 600′) may optionally comprise a gripping member (614, 614′) to provide additional grip when using the insertion tool to insert the anchor liner (200, 200′) into the bone (800) through rotation and/or longitudinal force.
      vi. Tensioning at least one end (401) of the suture (400) passed through the suture retention mechanism (616, 616′), or the at least one slit (607, 607′) of the suture retention mechanism (616, 616′), of the insertion tool (600, 600′). Optionally, each arm (606, 606′) may be a winged arm that curves proximally upward forming a valley adapted to receive an instrument (700) for tensioning the suture (400). The instrument (700) may receive, clamp, or grip the end (401) of the suture (400) and may optionally rest in the valley of the winged arm (606) when tensioning the suture (400) (see, for example, FIG. 47). This step may be repeated for each respective end (401) of the suture (400), such as in cases where two ends (401) of the suture (400) have each been received by the suture retention mechanism, or a respective slit (607, 607′) of the suture retention mechanism (616, 616′), of the insertion tool (600, 600′). The instrument (700) may optionally be used to gradually tension the suture (400) by progressively rotating, twisting, tightening, pulling, or tugging the suture (400) with the instrument (700). It should be understood that any instrument capable of holding and/or gripping the suture (400) may be used, including, for example, surgical instruments such as a hemostat, needle driver, clamp, or forceps. Alternatively, a user may tension the suture (400) directly themselves.
      vii. Removing the suture (400) from the insertion tool (600, 600′). This step may optionally include removing at least one end (401) of the suture (400) from the respective slit (607, 607′) of the respective arm (606, 606′), the respective first (609, 609′) or second (610, 610) channel, and, if applicable, the respective first (611) or second (612) suture hole of the insertion tool (600, 600′). This step may be repeated for each end (401) of the suture (400) received by a respective slit (607). In embodiments of the insertion tool (600′) having a suture alignment clip (650), the suture alignment clip (650) may need to be removed from the shaft (608′) of the insertion tool (600′) first in order to remove the suture (400) from the insertion tool (600′).
      viii. Removing the driving tip (613, 613′) of the insertion tool (600, 600′) from the cavity (207, 207′) of the anchor liner (200, 200′).
3. Securing the suture (400) by inserting an inner locking component (300, 300′) into an opening (208, 208′) of the cavity (207, 207′) of the anchor liner (200, 200′) by threading the inner locking component (300, 300′) into the cavity (207, 207′). An outer surface (304) of the inner locking component (300, 300′) may engage an inner wall (209, 209′) of the anchor liner (200, 200′) cavity (207, 207′). In some embodiments, a driving tool may be used to insert or drive the inner locking component (300, 300′) into the anchor liner (200, 200′) cavity (207, 207′).
4. Locking the suture (400) between the bone and the anchor liner (200, 200′) as the proximal end (201, 201′) of the anchor liner (200, 200′) progressively expands radially when the inner locking component (300, 300′) is inserted further into the cavity (207, 207′). One or more expansion areas (214, 214′) of the anchor liner (200) may be adapted to progressively expand radially the proximal end (201, 201′) of the anchor liner (200, 200′). In embodiments having a headless inner locking component (300′), the headless inner locking component (300′) may optionally be inserted into the cavity (207, 207′) of the anchor line (200, 200′) until a trailing end (302′) of the headless inner locking component (300′) rests flush with the proximal end (201, 201′) of the anchor liner (200, 200′). FIG. 49 depicts the suture anchor system (100) after it has been inserted into the bone (800) and the suture (400) has been secured.
5. In some embodiments, the method may optionally further comprise inserting an opposite end (401) of the suture (400) through an optional first suture channel (212, 212′) of a second anchor liner (200, 200′) of a second suture anchor system (100), into one opening (211, 211′) of a suture hole (210, 210′) and through the suture hole (210, 210′) to exit an opposite opening (211, 211′) of the suture hole (210, 210′), and through an optional second suture channel (213, 213′).
6. Inserting the second anchor liner (200, 200′) into a different location of the bone. In some embodiments, an external thread (206, 206′) of the second anchor liner (200, 200′) body (203, 203′) may engage the bone through rotation. In other embodiments, the external thread (206, 206′) may engage the bone through longitudinal force. In further embodiments, the external thread (206, 206′) may engage the bone through either rotation or longitudinal force. In at least some embodiments, an insertion tool (600, 600′) (see FIGS. 37-42 and 56-64) may optionally be used to insert and drive the anchor liner (200, 200′) into the bone (800), as described in Step 2(a).
7. Tensioning the suture (400) until the suture (400) is taut between the first and second anchor liners (200, 200′). In at least some embodiments, the insertion tool (600, 600′) may optionally be used to tension the one end of the suture (400) (see Step 2(a)(vi)).
8. Affixing the suture (400) by inserting an inner locking component (300, 300′) into an opening (208, 208′) of a cavity (207, 207′) of the second anchor liner (200) by threading the inner locking component (300, 300′) into the cavity (207, 207′). An outer surface (304, 304′) of the inner locking component (300, 300′) may engage an inner wall (209, 209′) of the anchor liner (200, 200′) cavity (207, 207′). In some embodiments, a driving tool may be used to insert the inner locking component (300, 300′) into the anchor liner (200, 200′) cavity (207, 207′).
9. Locking the suture (400) between the bone and the second anchor liner (200, 200′) as a proximal end (201, 201′) of the second anchor liner (200, 200′) progressively expands radially when the inner locking component (300, 300′) is inserted further into the cavity (207, 207′). one or more expansion areas (214, 214′) of the second anchor liner (200, 200′) may be adapted to progressively expand radially the proximal end (201, 201′) of the second anchor liner (200, 200′).

In at least one further embodiment, the method described above may comprise securing a suture (400) between one suture anchor system (100), as described, and one solid bodied anchor (not shown). In such embodiments, the solid bodied anchor does not comprise internal threads in a cavity and does not receive an inner locking component. In some embodiments, one end (401) of the suture (400) may first be secured to a bone (800) using the suture anchor system (100) according to Steps 1-4, and then the opposite end (401) of the suture (400) may be secured to the bone (800) using a solid bodied anchor according to Steps 5-7, wherein the solid bodied anchor does not include an inner locking component and only comprises an anchor liner without an expandable cavity. In other embodiments, one end (401) of the suture (400) may first be secured to a bone (800) using the solid bodied anchor according to Steps 1-2 (wherein the solid bodied anchor does not include an inner locking component and only comprises an anchor liner without an expandable cavity), and then the opposite end (401) of the suture (400) may be secured to the bone (800) using the suture anchor system (100) according to Steps 5-9.

Although described above in connection with securing a suture to a bone, these descriptions are not intended to be limiting, as other anchor systems and insertion tools can be made in accordance with the description herein, but of different size or scale, for use in other surgical procedures, as desired. As such, although the system and method are illustrated and described herein, various modifications and structural changes may be made therein without departing from the spirit of the system or method and within the scope and range of equivalents of the claims. Further, any steps described herein may be carried out in any desired order (and any additional steps may be added as desired and/or any steps may be deleted as desired).

Any reference in this specification to “one embodiment,” “an embodiment,” an “example embodiment,” etc., means that a particular feature, structure, or characteristic described in connection with the implementation is included in at least one embodiment of the system or method. The appearances of such phrases in various places in the specification are not necessarily referring to the same embodiment. In addition, any elements or limitations of any system or method or embodiment thereof disclosed herein can be combined with any and/or all other elements or limitations (individually or in any combination) or any system or method or embodiment thereof disclosed herein, and all such combinations are contemplated with the scope of the system and method without limitation thereto.

As used herein, the terms “substantial” and “substantially” refer to the complete or nearly complete extent or degrees of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context, which one of ordinary skill in the art would be familiar with. However, generally speaking the nearness of completion will be so as to have the same overall result as if absolute or total completion were obtained.

The use of term “substantial” or “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

Although described above in connection with particular configurations, these descriptions are not intended to be limiting as various modifications may be made therein without departing from the spirit of the system or method and within the scope and range of equivalents of the described embodiments. Encompassed embodiments of the present system and method can be used in all surgical applications calling for an anchor system or insertion tool for same.

While a number of embodiments of the present system and method have been described, it is understood that these embodiments are illustrative only, and not restrictive, and that many modifications may become apparent to those of ordinary skill in the art. For example, any element described herein may be provided in any desired size (e.g., any element described herein may be provided in any desired custom size or any element described herein may be provided in any desired size selected from a “family” of sizes, such as small, medium, large). Further, one or more of the components may be made from many different suitable materials.

In addition, various modifications and additions can be made to the exemplary embodiments discussed without departing from the scope of the present system or method. For example, while the embodiments described above refer to particular features, the scope of this system and method also includes embodiments having different combinations of features and embodiments that do not include all of the described features. Accordingly, the scope of the present system and method are intended to embrace all such alternatives, modifications, and variations as fall within the scope of the claims, together with all equivalents thereof.

What is claimed is:

1. A suture anchor system adapted for securing a suture to a bone, the system comprising:
an anchor liner comprising a substantially cylindrical elongated body having a proximal end, a distal end, and a longitudinal axis therebetween, the body further comprising:
an external thread on an exterior surface of the body;
a cavity bored through the body extending from the proximal end along the longitudinal axis, the cavity having an inner wall and an opening at the proximal end of the anchor liner,
a suture hole through the body transverse to the longitudinal axis and near the distal end of the anchor liner;
one or more expansion areas through a portion of the body at the proximal end; and
an inner locking component comprising a leading end, a trailing end and an elongated body therebetween, the trailing end adapted to receive a driving tool, the body having an outer surface adapted to engage the inner wall of the cavity.

2. The system of claim 1 wherein the proximal end of the anchor liner is adapted to progressively expand radially as the inner locking component is inserted into the cavity.

3. The system of claim 2 wherein the anchor liner is adapted to lock the suture between the bone and the anchor liner as the proximal end of the anchor liner expands.

4. The system of claim 1 wherein the one or more expansion areas comprise slits, expansion channels, slices, weakened or thinned areas, elastic areas, gaps, or perforated areas.

5. The system of claim 1 wherein the outer surface of the inner locking component further comprises an external thread and the inner wall of the cavity of the anchor liner further comprises an internal thread, the external thread of the outer surface adapted to engage the internal thread of the inner wall.

6. The system of claim 1 wherein the outer surface of the inner locking component further comprises an external thread and the inner wall of the cavity is smooth or textured, the external thread of the outer surface adapted to engage the inner wall.

7. The system of claim 1 wherein the outer surface of the inner locking component is smooth or textured and the inner wall of the cavity of the anchor liner further comprises an internal thread, the internal thread adapted to engage the outer surface.

8. The system of claim 1 wherein the outer surface of the inner locking component is textured and the inner wall of the cavity is textured, the textured outer surface adapted to engage the textured inner wall.

9. The system of claim 1 wherein the inner locking component further comprises a head at the trailing end.

10. The system of claim 1 wherein the inner locking component is headless.

11. The system of claim 1 wherein the exterior surface of the body of the anchor further comprises a threaded section and a smooth section, the threaded section comprising the external thread of the body and extending from the proximal end of the anchor liner partially along the body, the smooth section comprising the suture hole and extending from the distal end of the anchor liner to the threaded section.

12. The system of claim 1 wherein the external thread of the anchor liner is adapted to engage the bone through rotation.

13. The system of claim 1 wherein the external thread of the anchor liner is adapted to engage the bone through longitudinal force.

14. The system of claim 1 wherein the external thread of the anchor liner is adapted to engage the bone through either rotation or longitudinal force.

15. The system of claim 1 wherein the cavity of the anchor liner is adapted to receive a driving tip of an insertion tool.

16. The system of claim 1 wherein the body of the anchor liner further comprises first and second suture channels parallel to the longitudinal axis along opposite sides of the body, each suture channel recessed in the external thread of the body and extending from the proximal end to a respective opening of the suture hole.

17. The system of claim 16 wherein each of the one or more expansion areas of the body of the anchor liner is located radially between the first and second suture channels.

18. The system of claim 16 wherein the suture hole, the first suture channel, and the second suture channel are adapted to receive the suture.

19. The system of claim 1 wherein the body of the inner locking component is tapered or frustoconical in shape with a diameter narrowing gradually from the trailing end to the leading end.

20. The system of claim 1 wherein the body of the inner locking component is substantially cylindrical.

21. An insertion tool adapted for inserting an anchor liner of a suture anchor system into a bone and tensioning a suture therein, the insertion tool comprising:
a proximal end, a distal end and a body with a longitudinal axis therebetween;
the body comprising a handle and an elongated shaft extending from the handle to the distal end, the handle having a suture retention mechanism; and
the distal end comprising a driving tip extending distally outward and adapted to be received by the anchor liner.

22. The insertion tool of claim 21 wherein the suture retention mechanism comprises two arms each with a slit extending transverse to the longitudinal axis.

23. The insertion tool of claim 22 wherein the two arms are rotatably engaged with the body.

24. The insertion tool of claim 22 wherein each of the two arms is adapted to receive an instrument for tensioning the suture.

25. The insertion tool of claim 22 wherein each slit on each of the two arms is adapted to receive a respective end of the suture for tensioning.

26. The insertion tool of claim 21 wherein the driving tip is adapted to drive the anchor liner into the bone.

27. The insertion tool of claim 26 wherein the driving tip is adapted to drive the anchor liner into the bone through rotation or longitudinal force.

28. The insertion tool of claim 21 wherein the shaft of the body further comprises first and second channels along an exterior of the shaft, each channel on opposite sides of the shaft extending from the distal end and adapted to align with respective first and second suture channels of the anchor liner.

29. The insertion tool of claim 28 further comprising a suture alignment clip, the suture alignment clip comprising:
a substantially cylindrical hollow body having a proximal end, a distal end and a length therebetween;
an opening slit extending from the proximal end to the distal end for the length of the body, the opening slit having a width;
first and second side slits extending from the proximal end partially along the length of the body and located on opposite sides of the body; and
first and second protrusions on an interior of the body located on opposite sides of the body and extending from the distal end partially along the length of the body to the respective first and second side slits, the first protrusion aligning with the first side slit and the second protrusion aligning with the second side slit;
wherein the suture alignment clip attaches to the shaft at the distal end of the insertion tool; and
wherein the first and second protrusions are adapted to align with and slide into respective first and second channels on the shaft of the insertion tool.

30. The insertion tool of claim 29 wherein the first and second protrusions are each adapted to compress a suture received by each respective first and second channels of the insertion tool, and the first and second side slits are each adapted to allow an end of the suture to exit the suture alignment clip when the suture alignment clip is attached to the shaft at the distal end of the insertion tool.

31. The insertion tool of claim 21 wherein the shaft of the body further comprises first and second channels through the shaft, each channel extending from an opposite side of the shaft to respective first and second suture holes.

32. The insertion tool of claim 31 wherein the first suture hole and the second suture hole are adapted to align with respective first and second suture channels of the anchor liner.

33. The insertion tool of claim 31 wherein the first suture hole and the first channel, and the second suture hole and second channel are adapted to receive an end of the suture.

34. The insertion tool of claim 31 wherein the first and second suture holes are located at the distal end of the insertion tool.

35. The insertion tool of claim 34 wherein the driving tip is located between the first and second suture holes at the distal end of the insertion tool.

36. The insertion tool of claim 31 wherein the first and second channels and the respective first and second suture holes are adapted to maintain alignment of the suture when inserting the anchor liner into the bone.

37. The insertion tool of claim 21 further comprising a gripping member at the proximal end.

38. The insertion tool of claim 21 further comprising a surface at the proximal end adapted to receive a longitudinal force from a hammer or a mallet.
